(19) United States
(12) Patent Application Publication
(10) Pub. No.: US 2023/0390267 A1
(43) Pub. Date: Dec. 7, 2023

(54) TREATMENT REGIMENS FOR EARLY IDIOPATHIC PARKINSON'S DISEASE

(71) Applicant: BIAL - PORTELA & CA., S.A., S.Mamede Do Coronado (PT)

(72) Inventors: Patrício Manuel Vieira Araújo Soares Da Silva, S. Mamede Do Coronado (PT); José Francisco Da Costa De Pinho Rocha, S. Mamede Do Coronado (PT)

(21) Appl. No.: 18/201,920

(22) Filed: May 25, 2023

Related U.S. Application Data
(63) Continuation-in-part of application No. PCT/PT2021/050044, filed on Dec. 17, 2021.

(30) Foreign Application Priority Data
Dec. 17, 2020 (GB) 2019954.3
Apr. 29, 2021 (GB) 2106133.8
Jul. 7, 2021 (GB) 2109826.4

(51) Int. Cl. A61K 31/4439; A61K 31/198; A61P 25/16

(52) U.S. Cl. CPC A61K 31/4439; A61K 31/198; A61P 25/16

(57) ABSTRACT

Opicapone for use as adjunctive therapy to preparations of levodopa and a DOPA decarboxylase inhibitor (DDCI) in the treatment of Parkinson's disease; characterised in that a patient with Parkinson's disease is treatable with preparations of levodopa and a DDCI without clinically diagnosed motor complications.

### RELATED APPLICATIONS

This application is a continuation in part of International Application Serial No. PCT/PT2021/050044, filed Dec. 17, 2021, which claims the benefit of United Kingdom Patent Application Serial No. 2019954.3, filed Dec. 17, 2020, United Kingdom Patent Application Serial No. 2106133.8, filed Apr. 29, 2021 and United Kingdom Patent Application Serial No. 2109826.4, filed Jul. 7, 2021. The entire teachings of all of the aforementioned applications are incorporated herein by reference.

### FIELD OF THE INVENTION

This invention relates to treatment regimens for use in treating symptoms of early stage idiopathic Parkinson's disease. In particular, the invention relates to the use of opicapone as adjunctive therapy to levodopa and a DOPA decarboxylase inhibitor (DDCI) in the treatment of Parkinson's disease in a patient whose symptoms can be controlled with levodopa and a DDCI without motor complications.

### BACKGROUND OF THE INVENTION

Levodopa (L-DOPA) has been used in clinical practice for several decades in the symptomatic treatment of various conditions, including Parkinson's disease. Levodopa is able to cross the blood-brain barrier, where it is then converted to dopamine by the enzyme DOPA decarboxylase (DDC), thus increasing dopamine levels in the brain. However, conversion of levodopa to dopamine may also occur in peripheral tissues, possibly causing adverse effects. Therefore, it has become standard clinical practice to co-administer a peripheral DDC inhibitor (DDCI), such as carbidopa or benserazide, as adjunctive therapies. DDCIs prevent conversion of levodopa to dopamine in peripheral tissues. Levodopa/DCCI therapy remains the most effective treatment for the management of Parkinson's disease (Ferreira J, et al., Eur. J. Neurol., 2013; 20, 5-15).

During the early stages of Parkinson's disease, levodopa/DDCI therapy can almost entirely suppress symptoms of Parkinson's disease until the next dose is administered. However, most patients receiving long-term levodopa/DDCI will develop motor complications, such as end-of-dose motor fluctuations and dyskinesia, beyond the early stages of Parkinson's disease in spite of continued or increased levodopa administration (Aquino C C, Fox S H, Mov. Disord., 2015, 30, 80-89). Patients often report spending several hours per day with end-of-dose motor fluctuations in the so called “off” state and this can have a substantial effect on their quality of life (Chapuis S, Ouchchane L, Metz O, Gerbaud L, Durif et al., Mov. Disord. 2005, 20, 224-30). The development of motor complications, such as end-of-dose motor fluctuations, defines the transition from the early stage of Parkinson's disease to a more advanced stage of the disease. As such, the control of motor complications eventually becomes a key clinical need for almost all patients (Poewe W, Neurology, 2009, 72, S65-73).

End-of-dose motor fluctuations are linked to the short half-life of oral levodopa (about 60-90 min when administered with DDCIs). Catechol-O-methyltransferase (COMT) inhibitors increase the plasma elimination half-life of levodopa and decrease peak-trough variations and provide clinical improvements in Parkinson's disease patients afflicted with end-of-dose motor fluctuations. 2,5-dichloro-3-[5-(3,4-dihydroxy-5-nitrophenyl)-1,2,4-oxadiazol-3-yl]-4,6-dimethylpyridine 1-oxide (opicapone) is a potent and long-acting COMT inhibitor that reduces the degradation of levodopa to the inactive metabolite 3-O-methyldopa. Opicapone is bioactive, bioavailable and exhibits low toxicity. Thus, opicapone has potentially valuable pharmaceutical properties in the treatment of some central and peripheral nervous system disorders where inhibition of COMT may be of therapeutic benefit, such as, for example, mood disorders; movement disorders, such as Parkinson's disease, parkinsonian disorders and restless legs syndrome; gastrointestinal disturbances; oedema formation states; and hypertension.

Further research has focused on optimising opicapone into a stable and bioavailable form. For example, WO 2009/116882 describes various polymorphs of opicapone, with polymorph A being both kinetically and thermodynamically stable. WO 2010/114404 and WO 2010/114405 describe stable opicapone formulations used in clinical trials. WO 2013/089573 describes optimised methods for producing opicapone using simple starting materials and with good yields. The development of opicapone is described in L. E. Kiss et al, J. Med. Chem., 2010, 53, 3396-3411 and it was approved, in combination with levodopa and a DCCI, for the treatment of Parkinson's disease in the EU in June 2016, the US in April 2020 and Japan in June 2020 under the tradename “Ongentys”.

In all cases, opicapone is licenced as an adjuvant therapy to levodopa/DDCI preparations for use in patients beyond the early stages of Parkinson's disease. For example, the European label states: “Ongentys is indicated as adjunctive therapy to preparations of levodopa/DOPA decarboxylase inhibitors (DDCI) in adult patients with Parkinson's disease and end-of-dose motor fluctuations who cannot be stabilised on those combinations” (emphasis added). The US label states: “ONGENTYS is a catechol-O-methyltransferase (COMT) inhibitor indicated as adjunctive treatment to levodopa/carbidopa in patients with Parkinson's disease (PD) experiencing “off” episodes” (emphasis added).

The licencing of opicapone is based on the primary results from two pivotal phase III trials of opicapone in patients beyond the early stages of Parkinson's disease (i.e. in patients experiencing end-of-dose motor fluctuations). The trials are known as BIPARK-I (Ferreira et al., Lancet Neurol., 2016, 15, 154-65) and BIPARK-II (Lees et al., JAMA Neurol., 2017, 74, 197-206).

BIPARK-I demonstrated opicapone was superior to a placebo combined with levodopa/DCCI and non-inferior to previously-licenced COMT inhibitor, entacapone, in terms of its ability to reduce the time patients spent in the “off” state. BIPARK-II confirmed opicapone's efficacy and safety. These pivotal phase III trials confirmed the provisional results from smaller phase II trials. Post hoc analysis of the combined BIPARK studies and their open-label extensions suggest that opicapone also slows the rate of increase of time patients spend in the “off” state. In other words, opicapone appears to slow the progression of Parkinson's disease with respect to the levodopa needs in patients at more advanced stages of Parkinson's disease (WO 2016/083875), i.e. in patients experiencing end-of-dose motor fluctuations. It is important to remember that a treatment displaying therapeutic benefits at one stage of Parkinson's disease cannot be assumed to deliver the same benefits at another stage; indeed many do not.

A previously licenced COMT inhibitor, entacapone, was tested in patients suffering from early idiopathic Parkinson's disease, i.e. patients not suffering motor complications. The initial FIRST-STEP trial suggested that entacapone improved motor symptoms assessed by the Unified Parkinson's Disease Rating Scale (UPDRS) Parts II and III. However, the larger pivotal STRIDE-PD trial was unsuccessful in validating these provisional results. Thus, the use of entacapone as an adjunctive therapy to levodopa/DDCI treatment in early Parkinson's disease was not pursued. In fact, addition of entacapone was associated with a shorter time to onset of motor complications and increased frequency of dyskinesia. Therefore, COMT inhibitors are not currently recommended as adjunctive therapy to levodopa and a DDCI in the treatment of early Parkinson's disease, i.e. in patients whose symptoms can be controlled with levodopa and a DDCI with no motor complications.

There are currently three main classes of drugs considered suitable as monotherapy for people in the early stages of Parkinson's disease (Miyasaki J M, et al., Neurology, 2002, 58, 11-17; Fox S H, et al., Mov. Disord., 2011, 26, 52-41). The dopamine precursor, levodopa (combined with a DDCI) provides the greatest antiparkinsonian benefit for initial motor signs and symptoms, with the fewest side effects in the short term (Fox S H, et al., Mov. Disord., 2011, 26, 52-41; Olanow C W, et al., Mov. Disord., 2004, 19, 997-1005). As discussed above however, although it remains effective throughout the disease, it is associated with the development of motor complications (fluctuations and/or dyskinesia), typically necessitating the use of adjunct therapies to optimize the medication regimen. Therefore, the other classes of monotherapy are often preferred in the earliest stages of treatment. Monoamine oxidase (MAO)-B inhibitors (e.g. rasagiline, selegiline) prevent the breakdown of brain dopamine in the surviving dopaminergic neurons and can also be considered for the initial treatment of early disease (Rascol O, et al., Mov. Disord., 2016, 31, 1489-1496). However, because these agents typically offer mild symptomatic benefit, the majority of patients will require additional therapies for symptomatic efficacy within a relatively short timeframe (Olanow C W, et al., Mov. Disord., 2004, 31,1489-1496). Dopamine agonists (e.g. ropinirole, pramipexole, rotigotine) act directly on post-synaptic dopamine receptors and provide moderate symptomatic benefit. While their use as initial monotherapy can be used to delay the development of motor complications compared with levodopa, some patients suffer psychological or behavioural side effects (Antonini A, et al., Lancet Neurol., 2009, 8, 929-937).

As these treatments demonstrate, treatment of early Parkinson's disease is not simply a matter of increasing dopamine levels in the brain. In fact, excessive dosing with levodopa is directly associated with the development of motor complications in early Parkinson's disease (Stocchi F, et al., Ann. Neurol., 2010, 68, 18-27). But with disease progression, virtually all patients will require the superior symptomatic benefit/efficacy of levodopa. The fact that most Parkinson's disease therapies each have their own limitations at all and/or different stages of the disease has led to a stage-based approach to therapy in Parkinson's disease (Carrarini et al., Biomolecules, 2019, 9, 388) with levodopa/DDCIs being the gold standard despite its association with motor complications.

As such, general strategies that seek to increase the concentration or bioavailability of levodopa are not believed to benefit patients at the early stage of Parkinson's disease (i.e., before end-of-dose motor fluctuation symptoms appear) because any potential benefit when levodopa levels are low would be expected to be offset by increases in dyskinesia when levodopa levels are high (Stocchi F, et al., Ann. Neurol., 2010, 68, 18-27).

There remains a need for treatments that deliver and/or enhance symptomatic treatment in early Parkinson's disease. In particular, there remains a need for safe and effective treatment regimens that improve acute symptoms of early stage Parkinson's disease without inducing motor complications.

### SUMMARY OF THE INVENTION

The present inventors propose that opicapone can be used as adjunctive therapy to levodopa/DDCI in the treatment of Parkinson's disease in patients without motor complications.

Accordingly, in a first general embodiment, the invention provides opicapone for use as adjunctive therapy to preparations of levodopa and a DDCI in the treatment of Parkinson's disease; characterised in that the patient with Parkinson's disease is treatable with preparations of levodopa and a DDCI without clinically diagnosed motor complications.

In a second general embodiment, the invention provides the use of opicapone in the manufacture of a medicament for use as adjunctive therapy to preparations of levodopa and a DDCI in the treatment of Parkinson's disease; characterised in that the patient with Parkinson's disease is treatable with preparations of levodopa and a DDCI without clinically diagnosed motor complications.

In a third general embodiment, the invention provides a method of treating Parkinson's disease comprising administering opicapone as adjunctive therapy to preparations of levodopa and a DDCI to a patient in need thereof; characterised in that the patient with Parkinson's disease is treatable with preparations of levodopa and a DDCI without clinically diagnosed motor complications.

In a fourth general embodiment related to the first, second and third general embodiments, the administration of opicapone results in an improvement in one or more of the symptomatic readouts described below. Generally, the improvement in the patient is compared to symptoms that would be exhibited by a patient treated for the same period with preparations of levodopa and a DDCI without opicapone. Preferably, the treatment results in an improvement in one or more symptoms in the patient compared to the same patient prior to initiating opicapone treatment.

In a fifth general embodiment related to the first, second and third general embodiments, the administration of opicapone suppresses the emergence of one or more motor complications during treatment with opicapone and whilst maintaining levodopa/DDCI therapy.

### Rationale in Support of the Invention

The rationale underlying the present invention is set out in detail below. It supports a shift in the positioning of COMT inhibition with opicapone in the treatment of Parkinson's disease (PD) and lays out a pathway for proving its effectiveness in early disease.

In summary, the inventors propose that:
previous studies with tolcapone and entacapone were not viewed as unequivocally showing an effect in the treatment of PD in patients without motor complications, so failed to make an impact on product labelling and on how COMT inhibitors are used clinically;
the failure of entacapone to treat PD in patients without clinically diagnosed motor complications might be related to a poor understanding of its pharmacokinetic parameters at the time, coupled with the fact its administration is tied to the timing of levodopa dosing;
an important facet of opicapone's action in reducing motor complications in PD is associated with the avoidance of low plasma levodopa trough levels;
levodopa monotherapy does not address the problem of low levodopa trough levels and can instead worsen pulsatility and further affect basal ganglia output;
using opicapone to smooth out the delivery of exogenous levodopa in early disease potentially avoids exacerbating the already destabilized basal ganglia processing thereby preventing or delaying the emergence of motor complications;
based on this novel analysis of multiple physiological, pharmacological and clinical studies, opicapone is proposed as a novel option to treat PD patients without clinically diagnosed motor complications.

Levodopa is the most efficacious drug for the treatment of the motor symptoms of Parkinson's disease (PD) and the ‘gold standard’ therapy required by almost every patient affected by this common neurodegenerative disorder [1, 2]. However, its utility is often limited by the development of motor fluctuations (e.g. ‘wearing-off’, ‘ON-OFF’) and other motor complications (e.g. dyskinesia—chorea, dystonia, athetosis) [3]. While the incidence of troublesome dyskinesia appears to be declining with the more judicious use of levodopa [4], motor fluctuations, which can develop within a few years from treatment initiation, remains a common feature of PD. Motor fluctuations involve both motor and non-motor symptoms, that are under recognized by patients and underdiagnosed by physicians [5]. Modern cohort studies estimate the 5-year cumulative incidence of motor fluctuations ranges between 29-54% [6-8], increasing to 100% at 10 years [8] and, though the impact of motor fluctuations on daily life can be variable [9], numerous studies consistently show that they have a detrimental impact on quality of life [10-13], with their effective management remaining a significant unmet need [3, 14]. This is illustrated by the fact that once motor fluctuations develop, cumulative daily OFF time can account for up to 50% of a patient's waking day [15]. Indeed, wearing-off is reported by patients as a more significant and inconvenient component of current treatment than non-troublesome dyskinesia [16].

Even today, definitions of ON and OFF remain a matter of debate. Individual physicians use differing terminology, with the term ‘wearing-off’ being used to cover a variety of circumstances related to inadequate levodopa dosing, end of dose deterioration or ON-OFF phenomena. However, a good working definition of the wearing-off phenomenon would be a decrease in the duration of effect of each individual dose of levodopa with increasing disease progression and duration of drug treatment. While often held to be a complication of later disease, there is compelling evidence that it can emerge within months of starting levodopa therapy, but despite intense research, the pathophysiological mechanisms responsible for wearing-off remain unclear and patient risk factors poorly defined [17-19].

Pharmacodynamic factors involving both presynaptic and post-synaptic changes in dopaminergic and basal ganglia function appear responsible as opposed to any change in the peripheral pharmacokinetic profile of levodopa. Nevertheless, even with the uncertainties, it is well accepted that a key contributing factor to the development of wearing-off is the short plasma half-life of levodopa, leading to a non-physiological, ‘pulsatile’ stimulation of striatal dopamine receptors, which in turn is thought to result in a disorganised striatal output and disruption of the motor programs that control voluntary movement [20, 21].

Most current pharmacological strategies to improve motor function are based on the premise that there is inadequate and discontinuous stimulation of striatal post-synaptic dopamine receptors and that providing more continuous dopaminergic stimulation leads to an increase in ON time. In clinical practice, physicians employ a range of strategies to try to improve levodopa delivery to brain and to maintain dopamine receptor stimulation. These can include levodopa modification strategies such as increasing the levodopa dosage, increasing oral levodopa dosing frequency, and the use of controlled or sustained release preparations of the drug. While these levodopa approaches are inexpensive and usually effective in the short-term, they do not address the problem of low levodopa trough levels and can instead worsen pulsatility and further affect basal ganglia output. Continuous intra-duodenal delivery of levodopa is often highly effective, but is invasive and cannot be employed in all patients [22, 23]. An alternative is to use a longer acting oral dopamine agonist drug, such as ropinirole or pramipexole, to provide more continuous receptor stimulation [24, 25] or to deliver a dopamine agonist by subcutaneous infusion or transdermal administration as in the case of apomorphine and rotigotine [26, 27]. However, dopamine agonists bring other potentially significant adverse events (such as hallucinations, confusion and impulse control disorders) into the risk-benefit equation and are therefore not usually employed in an older PD population [28]. Recently, non-dopaminergic approaches to altering basal ganglia function have been suggested as effective in improving wearing-off such as the adenosine A_{2A} antagonist, istradefylline [29] and the NMDA antagonist, amantadine [30].

Like dopamine agonists, while these non-dopaminergic approaches usually reduce the severity of fluctuations, they do not affect the pharmacokinetic profile of levodopa and therefore do not address the underlying problem.

One tried and tested strategy has proved consistently effective in enhancing the plasma profile of levodopa and its delivery to the brain and the duration of effect of each dose—and that is through the use of enzyme inhibitors controlling the activity of the key catabolic pathways that determine the efficacy of levodopa. The first of these were the peripheral decarboxylase inhibitors, carbidopa and benserazide, which are used as standard to increase levodopa availability to brain at all stages of the disease. Subsequently, the irreversible monoamine oxidase B inhibitors, selegiline and rasagiline were developed and are now commonly used as early monotherapy and as adjuncts to levodopa to prolong the duration of effect of endogenous dopamine and dopamine formed from levodopa in brain [31]. More recently, the reversible MAO-B inhibitor safinamide has also been introduced into therapy as an adjunct to levodopa [31].

The COMT inhibitors, entacapone, tolcapone and opicapone were specifically developed for the management of wearing-off as they act to protect levodopa from its major peripheral pathway of metabolism by the COMT enzyme. Although tolcapone has been shown to inhibit central COMT, its clinical efficacy seems to be mainly mediated through inhibition of peripheral COMT and depends on concomitant use of exogenous levodopa [32]. Tolcapone and entacapone were introduced in the 1990's, and have mainly been used for more advanced patients with chronic motor fluctuations. However, neither compound has turned out to be ideal—tolcapone being associated with hepatic toxicity and entacapone having a short plasma half-life requiring dosing with every administration of levodopa [33]. Opicapone is a third generation COMT inhibitor rationally designed to reduce the risk of toxicity and improve COMT inhibitory potency and peripheral tissue selectivity compared with other COMT inhibitors [34]. It was first approved in Europe for the management of motor fluctuations in 2016, and since been approved for use in the USA, Japan, South Korea, Australia and other countries. Despite its obvious advantages over earlier COMT inhibitors, opicapone has also been largely reserved for use in later stage patients with wearing-off where other treatment strategies have failed.

1. Peripheral Enzyme Inhibition is an Essential Factor that Determines the Effect of Levodopa in Brain

The combination of levodopa with a DDCI is now so embedded in clinical practice in PD that “levodopa monotherapy” inevitably means levodopa plus a DDCI, and nobody would contemplate using levodopa without a DDCI from the very start of treatment.

However, using levodopa with a DDCI does not overcome many of the inherent problems in its use. The oft quoted ‘short’ 90 minute half-life of levodopa, actually refers to the plasma pharmacokinetics of oral levodopa combined with carbidopa [44] and the extent of brain penetration of levodopa remains low, reaching only 10% when combined with a DDCI. A major reason for these continued deficits in levodopa's profile is linked to its other pathway of metabolism—namely through COMT. COMT is another ubiquitous enzyme found in the periphery and brain and is responsible for the O-methylation of a wide range of catechol-containing substrates. In peripheral tissues, COMT is mainly available in its soluble cytosolic form (S-COMT) with the highest activities being described in the liver, kidney and gastrointestinal tract, whereas its membrane bound form (MB-COMT) predominates in the CNS [45]. As a consequence, peripheral COMT inactivates much of each levodopa dose before it can cross into the brain. Indeed, COMT converts about 90% of levodopa to 3-O-methyldopa (3-OMD) which in contrast to levodopa itself, has a long plasma half-life and accumulates on repeated levodopa administration as it is not a substrate for DDC. While no adverse effects of 3-OMD have been reported, it may compete with levodopa for transport in to brain at the level of the blood-brain barrier [46]. It is an underappreciated fact that, when a peripheral DDCI inhibitor is used, levodopa metabolism is shunted to the COMT pathway (and increases the formation of 3-OMD) such that only 5 to 10% of the administered drug reaches the brain [47].

The logical consequence of needing to block both peripheral DDC and peripheral COMT to maximize the effect of levodopa in PD was recognized early on, but the concept proved difficult to translate into a viable medication. Early attempts to inhibit COMT using compounds such as pyrogallol showed these molecules to be non-specific, inhibiting a range of enzyme systems and, more importantly, to be short acting and toxic [48]. Only with the discovery of the nitrocatechols (the ‘capone’ series) did the clinical reality of selective COMT inhibition in PD start to appear. One of the first to be developed was nitecapone which was effective, but showed toxicity that prevented clinical development [49]. Tolcapone was a useful and effective drug and was registered for use in treating levodopa wearing-off, but the subsequent discovery of its potential for liver damage limited its use with extensive monitoring required—despite subsequent extensive safety studies showing the usefulness of the compound [50, 51]. Entacapone was also successfully registered for the treatment of PD, but since its half-life was as short as that of levodopa, the use of the two drugs had to be linked to achieve a successful inhibition of COMT. This practical limitation was overcome to some extent by the introduction of Stalevo as a combined levodopa/carbidopa/entacapone combination, but use of this “triple combination” limits a physicians' ability to tailor levodopa dosing to a patient's individual needs. Consequently, whilst Stalevo was developed for improved medication compliance, it can be difficult to use in patients who are on differing levodopa doses at different times of the day and with complicated dosing regimens. Moreover, while entacapone had some effect in improving the pulsatility of the levodopa plasma profile, it was less effective than tolcapone and the peaks and troughs in levodopa plasma levels were still marked. Thus, while the second generation COMT inhibitors did start to address the pharmacokinetic limitations of levodopa, by inhibiting its peripheral metabolism and increasing levodopa delivery to the brain, they did not solve the problem of optimizing levodopa delivery.

2. Opicapone—Experimental Biochemistry and Pharmacology

The search for a once daily, potent, selective and long acting peripheral COMT inhibitor lacking toxicity culminated in the development of opicapone as a third generation molecule for the treatment of PD. Opicapone was designed as a 1,2,4-oxadiazole analogue with a pyridine N-oxide residue at position 3 and so is chemically distinct from the previous generation of nitrocatechols. Its unique pharmacophore resulted in high COMT inhibitory potency in the absence of cellular toxicity [52]. In addition, opicapone has sub-picomolar binding affinity to S-COMT in peripheral tissues and does not appear to have any effect on COMT activity in brain [52]. Opicapone does have a relatively short plasma half-life and would not immediately be expected to produce a long-lasting inhibition of COMT. However, its binding and interaction with S-COMT is prolonged and outlasts the clearance of the drug from the systemic circulation. Roughly translated, opicapone tightly binds to S-COMT, but it is a poor substrate and therefore inactivates enzyme activity for a prolonged period [53]. The tight binding and slow complex dissociation characteristics of opicapone are fundamental to its COMT inhibitory potency and once-daily dosing frequency.

The persistent enzyme inhibition produced by opicapone translates into functional activity that can be seen both in vitro and in vivo in experimental models. In liver and kidney homogenates from rats treated orally with opicapone, tolcapone or entacapone, opicapone produced a more marked and more sustained inhibition of COMT than the other drugs [4, 55]. The effects on levodopa (in conjunction with a DDCI) metabolism also reflects the long-lasting inhibition of COMT produced by opicapone. Oral administration of opicapone with levodopa to rats resulted in a sustained increase in brain levodopa levels that was evident 24 hours after drug administration. Similar results were seen in the cynomolgus monkey, where administration of adjunct opicapone to levodopa/benserazide increased levodopa systemic exposure by 2-fold not changing Cmax values [56, 57] and reduced both 3-O-methyldopa (3-OMD) exposure and Cmax values by up to 7-fold [, 57]. These changes were accompanied by an up to ˜85% reduction in erythrocyte COMT activity [, 57] and translated into an improvement in motor function in MPTP treated parkinsonian primates [57].

3. Effect of Opicapone on Levodopa Pharmacokinetic Profile

Similar to in vitro and in vivo experimental models, the pharmacokinetics of opicapone in man would not initially seem consistent with a drug for once daily administration. Single oral doses of opicapone ranging from 10 to 1200 mg administered to healthy male volunteers showed dose proportional exposure to the drug in plasma and a terminal elimination half-life of opicapone of between 0.8 to 3.2 hours. However, the duration of COMT inhibition by opicapone was independent of the dose and the half-life of COMT inhibition in erythrocytes was 61.6 hours, reflecting the estimated dissociation of the COMT-opicapone molecular complex. Thus, despite a relatively short plasma half-life, opicapone markedly and sustainably inhibited peripheral S-COMT activity long after its plasma clearance [59, 60]. This long duration inhibition of COMT is reflected in changes in the plasma kinetics of levodopa. In patients with PD, administration of opicapone dose dependently increases levodopa bioavailability by up to 65% dependent on dose and duration of drug administration [40,43]. As assessed by AUC, opicapone was more effective in increasing levodopa exposure than occurred after entacapone administration, reflecting its sustained COMT inhibition that endures over 24 hours [59]. Administration of opicapone also increased the minimum plasma concentration (Cₘᵢₙ) for individual levodopa doses by up to 2.6-fold. This is an important facet of opicapone's action as a reduction in motor fluctuations in PD is associated with the avoidance of low plasma levodopa trough levels [61].

There may also be another advantage of the dissociation between opicapone's pharmacokinetic profile and its functional activity. In some PD patients, entacapone absorption interferes with levodopa absorption resulting in a delayed levodopa tₘₐₓ and reduced Cₘₐₓ on simultaneous administration [62, 63]. This might explain an apparent lack of response to entacapone in some individuals [62]. While there is also a potential interaction between levodopa and opicapone when given at the same time, its once daily bedtime administration (at least one hour before or after levodopa combinations) and rapid plasma clearance minimizes any interaction with levodopa based on drug absorption [64]. Thus, the pharmacokinetic profile of opicapone, and its subsequent effect on levodopa availability, provides a treatment strategy based on once daily administration at a single effective dose where inhibition of COMT is not tied to the timing of levodopa dosing or to any specific levodopa product or dose—as is the case with entacapone. It is also easier and more convenient for patient compliance and drug cost. Recently, the UK National Institute for Health and Care Excellence (NICE) highlighted that using a once-daily administration of opicapone enables flexible dosing of levodopa without altering the opicapone dose [65].

4. Efficacy in Fluctuating Parkinson's Disease

4.1 Rationale for COMT Inhibition in the Management of Motor Fluctuations

There is a clear and obvious rationale for using a peripherally acting COMT inhibitors in patients treated with levodopa where motor complications have appeared. The troughs in plasma levodopa levels that occur between doses directly correspond with OFF symptoms [61, 66] and the treatment objective with a COMT inhibitor is to maintain levels above the threshold for ON. Adding a COMT inhibitor to the levodopa regimen helps to extend the benefit of each levodopa dose and avoid the fluctuations associated with oral levodopa therapy without unnecessarily increasing the dose or frequency of levodopa administration [67]. The shunting of levodopa metabolism to the COMT pathway is avoided and results in increased drug exposure. So, by inhibiting both the major pathways of levodopa metabolism through the use of a DDCI and a COMT inhibitor, the delivery of levodopa to the brain can be maximized and more continuous drug delivery achieved.

4.2 Clinical Studies of Opicapone in Patients with Wearing-Off

The efficacy of adjunct opicapone in reducing OFF time in patients with motor fluctuations has been well established in phase 3 clinical trials as well as in observational studies and has been extensively reviewed elsewhere [68, 69]. Three randomized, double-blind, placebo-controlled studies have examined the symptomatic effects of opicapone in PD patients with motor fluctuations, namely the BIPARK studies (I and II) [70, 71] and, more recently, a phase 2b (COMFORT-PD) study conducted in the Japanese population [72].

In a pooled analysis of the BIPARK studies, double-blind treatment with opicapone (25 and 50 mg) significantly reduced absolute daily OFF-time. The mean [95% CI] treatment effect versus placebo was −35.1 [−62.1 , −8.2] minutes (p=0.0106) for the opicapone 25 mg dose and −58.1 [−84.5 , −31.7] minutes (p<0.0001) for the 50 mg dose [73]. A statistically significant increase in ON time without dyskinesias was also observed, while troublesome dyskinesias did not increase [73]. Such results were replicated in the Japanese study, which showed a placebo adjusted reduction in OFF time of −0.74 hours with the opicapone 25 mg dose group and −0.62 hours with the 50 mg dose (p<0.05 for both opicapone groups). OFF-time was consistently and steadily reduced in both opicapone tablet groups from Week 1 up to the end of the double-blind part (14-15 weeks) [72].

While the BIPARK I study was not designed to test for superiority of opicapone to entacapone (rather it tested for non-inferiority), statistically significant improvements were also seen in CGI-C and PGI-C scores for opicapone 50 mg versus the active comparator entacapone [70]. Placebo-adjusted OFF time reductions in the study for entacapone (−40.3 minutes) were entirely consistent with prior studies (a meta-analysis of entacapone studies reports a reduction of −41 minutes [74]) and the treatment difference for opicapone 50 mg versus entacapone of 26.2 minutes bordered statistical significance (p=0.05) [70]. Based on these data, the opicapone levodopa-equivalent dose (LED) was recently estimated to be 1.5, which is the same as for tolcapone and higher than the entacapone LED conversion factor of 1.3. Thus, opicapone's LED is 140-150 mg for a 100 mg levodopa dose [75]. Clinical differences between the products are further highlighted by the switch from entacapone in BIPARK I to opicapone in the open label extension. Patients previously treated with entacapone in the double-blind phase had an average reduction of 40 minutes OFF-time, and subsequently experienced an additional improvement of 68 minutes OFF-time reduction in patients that ended open-label with opicapone 50 mg treatment [76]. A similar hint of improved efficacy was reported in an audit of previous entacapone users at a single UK site [77]. The audit included 20 patients who switched from entacapone to opicapone and 37 patients who had previously experienced a lack of efficacy or adverse events with entacapone. In those patients who continued with opicapone beyond 6 months, the mean reduction in OFF time of was reported to be −2 hours per day as measured by interview.

Patients who switched from entacapone to opicapone were more likely to remain on opicapone treatment than those who had previously experienced AEs with COMT inhibition [77]. The superior efficacy of tolcapone to entacapone is well known [78], but tolcapone's safety profile means that it can be only considered after entacapone [79]. Opicapone has no such restrictions and its once daily dosing together with indications for higher efficacy than entacapone has led to the suggestion that it is a good alternative to entacapone in patients with motor fluctuations [69].

Moving to the real-world observational setting, Reichmann and colleagues conducted the OPTIPARK study which included 506 patients treated at 68 centers in the UK and Germany [80]. After 3 months of treatment with opicapone 50 mg, the majority of patients (71.3%) showed clinical improvement as judged by the investigators (CGI-C), with 43% reported as much or very much improved. For those UK patients (n=95) who were also assessed at 6 months, 85.3% were judged as improved since commencing treatment (including 8.4% who were very much improved and 49.4% who were much improved) while 8.4% were judged as showing ‘no change’ and 6.4% as having worsened. Importantly, this high opinion of efficacy was corroborated by the patients themselves, of whom 76.9% reported they were improved at 3 months. Even though patients came into the study on optimized therapy (79% of patients were receiving levodopa plus another PD medication), the study found that opicapone adjunct therapy was associated with clinically relevant improvements in UPDRS motor and ADL scores (by 4.6 and 3.0 points, respectively) [80]. These changes within the range of estimated clinically relevant differences of 2.0-5.2 points (for motor scores) and 0.5-2.3 points (for ADL scores) [81], indicating that treatment with opicapone not only increases ON time, but also improves the quality of ON time.

Another interesting finding is that after 3 months of treatment with opicapone, most patients remained on the same total daily levodopa frequency (77.1% had no change, 10.4% had an increase and 12.5% had a decrease in dosing frequency), resulting in an overall mean reduction of approximately −10 mg/day. This is consistent with the pivotal studies where, for example, in the BIPARK II study almost two-thirds (63%) of patients were maintained on the same dose of levodopa, despite freedom to adjust dosing according to clinical need [71]. The average number of daily levodopa intakes also remained stable during this phase, ranging from 4.7 to 4.8 over one year. In all studies, the most common reason for reducing the levodopa dose is to manage dopaminergic adverse events such as dyskinesia, while the maintenance of levodopa dosing with opicapone in the OPTIPARK [80] and the year-long open-label extensions of the pivotal studies [62, 73, 82] hint at a possible long-term delay of need for levodopa increases. In fact, this concept will be further explored in an ongoing study of early wearing-off.

4.3 Opicapone in Early Wearing-Off

It is increasingly accepted that motor fluctuations start to emerge much earlier than once thought [21], with wearing-off already common within the first few years from treatment, and underestimated by routine neurological clinical evaluation [83]. Reasons for under recognition of wearing-off include a lack of appreciation of non-motor fluctuations as well as a general lack of patient (and physician) awareness of the phenomenon [84]. With continued medical education these issues are slowly being addressed [85], but there is still a particular tendency for neurologists to underestimate the presence of wearing-off in patients within the first few years of diagnosis (<2.5 years disease duration) perhaps as they wait to have a more objective picture of established symptom re-emergence [83]. Predictors of wearing-off (in addition to duration of treatment and disease severity) include a younger age, weight and female gender [18, 83], with women showing an 80% increased risk for wearing-off [86].

Taken overall, patients enrolled into the BIPARK studies had a disease duration of almost 8 years, motor fluctuations for almost 3 years and a mean daily OFF time of over 6 hours [73]; the majority (83%) of patients received polypharmacy (levodopa plus at least one other PD medication) for their parkinsonian symptoms [87]. While this might indicate a population with more chronic motor complications, the studies did include patients with lesser amounts of OFF time at baseline as well as patients who were not on other adjunct therapies. Recent post-hoc analyses of patients with wearing-off who were earlier in their PD and treatment journey indicate equivalent, and even enhanced, efficacy of opicapone 50 mg vs placebo when compared to the overall pooled population [88]. For example, patients with a more recent onset of motor fluctuations (within the previous 2 years) had a placebo-adjusted reduction of OFF time of −68.5 minutes (p=0.0003 vs. placebo) and patients taking less than 600 mg of levodopa per day had a placebo-adjusted reduction of OFF time of −75.5 minutes (p=0.0005 vs. placebo) [73, 88]. Such OFF time reductions are, again, likely to be clinically relevant as they exceed the estimated clinically relevant difference of 1 hour [8, 89]. Although the sample size was relatively small (n=67 in the opicapone 50 mg group and n=59 in the placebo group), patients only taking levodopa at baseline (i.e. without dopamine agonists or MAO-B inhibitors as adjunct therapy) also showed a placebo-adjusted mean reduction of −65.6 minutes (p=0.02 vs placebo) with opicapone 50 mg, supporting its utility as soon as wearing-off symptoms appear.

Finally, there is some evidence that earlier versus later initiation of opicapone may be beneficial for patients with motor fluctuations. In a combined analysis of the BIPARK double-blind and open-label studies, OFF-time reductions at the end of the open-label phase were found to be numerically greater for the patients assigned to opicapone in the double-blind phase versus those who were originally assigned to placebo (change from baseline of −141.1 minutes in the group that received opicapone 50 mg throughout double-blind and open-label treatment versus 114.7 minutes in the group that switched from placebo to opicapone) [73]. A similar tendency for the earlier (versus 6 months later) initiation of entacapone has previously been shown using data from another pooled analysis of placebo-controlled trials and open-label extensions [90] and has led to the suggestion that there may beneficial effects of earlier versus later initiation of COMT inhibitors in subjects with levodopa-related fluctuations. Whilst the participant numbers dropped by over 90% by the time a difference was detected at 4-5 years from baseline, this concept merits further prospective study.

As stated above, the standard treatment approach to wearing-off is to alter the dosing regimen of conventional levodopa formulations, either by increasing the size of each levodopa dose or by “fractionating” the total daily levodopa dose into smaller, more frequent doses (Brooks D J. Neuropsychiatr. Dis. Treat.; 4(1): 39-47; 2008). Putting all pieces together from numerous physiological, pharmacological and clinical studies, opicapone once-daily could be considered a potential first line adjunctive levodopa therapy to treat wearing-off that could, potentially, even limit the need to increase the amount of levodopa required in the long run. A randomized, parallel group, multicentre, multinational, prospective, open-label exploratory clinical study (eArly levoDopa with Opicapone in Parkinson's paTients wIth motOr fluctuatioNs [ADOPTION] study; EudraCT number 2020-002754-24) is currently underway to evaluate the effect of opicapone 50 mg in PD patients with early wearing-off. In this study, patients (aged 30 years or older) with idiopathic PD, treated with 3-4 daily oral doses of up to 600 mg levodopa, with signs of treatable motor disability and experiencing wearing-off phenomenon for less than two years will be randomized (1:1 ratio) to receive opicapone 50 mg once-daily or an extra dose of 100 mg levodopa during a 1-month evaluation-period. Efficacy endpoints will be based on patients home diaries [91], as well as the Movement Disorder Society-Unified Parkinson's Disease Rating Scale (MDS-UPDRS) [92], the Movement Disorder Society-Non-Motor Rating Scale (MDS-NMS) [93], the Parkinson's Disease Questionnaire-8 (PDQ-8) [94], the Clinical Global Impression of Improvement (CGI-I) and the Patient Global Impression of Change (PGI-C) [95].

5. Efficacy in Stable Parkinson's Disease

5.1 A Rationale for COMT Inhibition in Early ‘Stable’ Disease

There are two reasons to consider COMT inhibition in stable disease, i.e. before the development of motor complications. The first is to prevent or delay the development of motor fluctuations, and the second is to alleviate current symptoms in a stable patient. In this context, ‘stable’ disease refers to the period of time when patients are enjoying the benefits of their levodopa therapy without diagnosed motor complications. In other words, what has long been termed the ‘honeymoon’ period. It is not quite the same as (but often confused with) ‘early’ disease which is often used to refer to the first few years' post-diagnosis. As previously mentioned, a proportion of patients develop motor complications fairly early on in the course of their disease.

Whereas the rationale for COMT inhibition in managing motor fluctuations is easy to understand, the rationale for preventing or delaying the emergence of motor fluctuations requires a deeper understanding of how the basal ganglia strives for equilibrium. At the turn of the century, there was an explosion of work to understand the impact of levodopa pharmacokinetics and the ‘pulsatile’ delivery associated with intermittent oral administration of levodopa/DDCI. In stable disease, a key reason for providing a more continuous drug delivery (CDD) is that it will provide a more continuous dopaminergic stimulation (CDS). The preclinical and clinical evidence for both concepts has been extensively reviewed elsewhere [21, 67, 96-98]. While the concept of CDS is quite complex, the very basic premise is that, under normal physiological conditions dopaminergic neurons originating from the substantia nigra fire tonically (independently of movement), producing a steady baseline concentration of extracellular dopamine in the striatum. This sustains a background level of continuous stimulation of striatal dopamine receptors, with phasic dopamine release occurring in response to behavioral activity. In the normal brain, presynaptic vesicular storage of dopamine acts as a transmitter reservoir, thereby providing a natural buffer to ensure the constant stimulation the striatum expects. With nigrostriatal degeneration, this buffering capacity is progressively lost. In the short-term, this leads to abnormal patterns of striatal function, including the non-physiological modulation of corticostriatal glutamate release by dopamine. In the long-term, physiological consequences include an abnormal plasticity of corticostriatal synapses leading to a profound destabilization of striatal output, downstream molecular and neurophysiological changes in the rest of the basal ganglia (including changes in long-term potentiation [LTP] and depression [LTD]), and, ultimately, alters the way the basal ganglia processes motor information [99].

Under these conditions, the way levodopa is delivered to replace the endogenous dopamine is believed to be important. Given its short half-life, oral administration is associated with peaks and troughs of levodopa—and, therefore, exogenous dopamine-availability. This pattern of delivery does not reflect the physiological tonic stimulation that occurs in the normal brain and leads to a further perturbation of basal ganglia processing, ultimately manifesting as the motor complications of wearing-off and dyskinesia. The inventors believe that using a COMT inhibitor to smooth out the delivery of exogenous levodopa in early disease will avoid exacerbating the already destabilized basal ganglia processing thereby preventing or delaying the emergence of motor complications.

5.2 Clinical Studies of COMT Inhibition in ‘Stable’ Patients

The failure of the STRIDE (STalevo Reduction In Dyskinesia Evaluation) study to show a delay in dyskinesia development with early use of the levodopa/carbidopa/entacapone (Stalevo) combination initially suggested that there was no advantage to early COMT inhibition [100]. However, the inventors believe that the STRIDE study was flawed in a number of ways. A primary deficit was that the study was initiated on the basis of findings in MPTP marmosets who were dosed four times a daily at 3.5 hourly intervals [101]. Pharmacokinetic studies in man were only conducted later [67, 102, 103] and the dosing interval was shown not to produce CDD [18, 102, 103]. Furthermore, these pharmacokinetic studies showed that repeated entacapone dosing increased levodopa Cₘₐₓ values in plasma which would increase the risk of dyskinesia development. Criticism can also be made of using levodopa dosing schedules that increased it up to 400 mg/day in the first year of treatment which contrasts with normal clinical practice in this period. The inventors believe that a better understanding of these pharmacokinetic parameters at the time, would have led to the design of STRIDE being very different.

In contrast, treatment of patients with PD with once daily opicapone 50 mg increased systemic exposure to levodopa given every 3 and 4 hours, leading to both decreased peak-to-trough fluctuations in levodopa concentrations and to higher trough levodopa concentrations [104]. Based on this, the inventors believe that opicapone can provide the level of CDD required to avoid dyskinesia if started in early disease. Although the concept of CDD with opicapone could be examined in a pharmacokinetic study, the clinical translation into reduced dyskinesia is probably unlikely to be tested in a formal STRIDE-like study, which needs to be very large, at least 2-4 years long, and therefore difficult to recruit for and expensive to conduct.

Another important question is whether COMT inhibition would help to further alleviate motor symptoms in a ‘stable’ patient (i.e. a patient for whom a complete response to levodopa treatment is possible without the presence of motor complications). In an early tolcapone study, 6 months treatment with tolcapone at 100 or 200 mg three times daily produced significant reduction in the UPDRS Part II activities of daily living (ADL, −1.4 & −1.6 points, respectively) and motor (−2.0 & −2.3 points, respectively) scores in ‘stable’ patients. These improvements were maintained up to the 12-month assessment and fewer patients in the tolcapone groups than in the placebo group developed motor fluctuations during the trial [105]. Likewise, in the FIRST-STEP study a significant difference in total UPDRS scores in favor of Stalevo was first observed at week 4 and was maintained through the 39-week observation period, with the greatest difference occurring at week 26 [106]. Similar findings have also been hinted at in prior studies with separate entacapone, which showed that adding a COMT inhibitor to the levodopa regimen in subgroups of stable patients improved scores, while maintaining levodopa dose levels over 6 months (in contrast to increased levodopa dosing in the placebo group) [107, 108]. These studies also showed that the benefit gained with entacapone in UPDRS scores was consistently lost when the drug was withdrawn [107, 108]. However, these studies have not been viewed as unequivocally showing an effect in stable disease, and as such, they have failed to make an impact on product labelling and on how COMT inhibitors are used in PD.

In contrast, the inventors believe that the unique profile of opicapone makes it an excellent candidate to test for benefit in ‘stable’ patients.

A randomized, double-blind, placebo-controlled, clinical study (Early ParkinSon wIth L-DOPA and OpicapoNe [EPSILON] study; EudraCT number 2020-005011-52) was designed to evaluate the effect of opicapone 50 mg in ‘stable’ PD patients. In this study, patients (aged 30-80 years) with idiopathic PD, treated with 3-4 daily oral doses of up to 500 mg levodopa, with signs of treatable motor disability but no motor complications were randomized in a 1:1 ratio to receive opicapone 50 mg once-daily or placebo during a 6-month double-blind evaluation-period. The patients' current levodopa/DDCI regimen remained stable throughout the double-blind period. The primary endpoint was the change from baseline to end of double-blind period in MDS-UPDRS Part III (motor) scores, and secondary outcomes assessed non-motor symptoms, quality of life and global clinical impressions of change. These data are discussed below. At the end of the double-blind period, patients may enter an additional 1-year, open-label period of opicapone 50 mg treatment [109].

6. Non-Motor Efficacy of Opicapone

A relatively unexplored dimension of efficacy is the impact of adjunct opicapone on non-motor symptoms. In the BIPARK II study, non-motor symptoms were assessed by the Non-Motor Symptoms Scale (NMSS) at different time points, including baseline, end of the double-blind phase and end of the open-label phase. At the end of the double-blind period, NMSS scores slightly improved across the opicapone and placebo groups, with no significant differences between them. At the 1-year open-label endpoint, a mean improvement of −4.2 in NMSS total score was still maintained [71]. No deterioration of any particular domain was observed and it is important to highlight that there was no worsening of dysautonomia, hallucinations or cognitive dysfunction.

Total NMSS scores are hard to interpret as the construct mixes together non-motor items that can be improved or worsened by dopaminergic agents at the same time. More interesting, however, was the significant signal seen for the sleep/fatigue domain where the 50 mg dose reduced the NMSS sleep/fatigue score by −1.2 points versus −0.5 points with placebo (p>0.05). Such benefits in non-motor scores, including sleep/fatigue, were also seen in the OPTIPARK study, where the mean±SD improvements of −6.8±19.7 points for NMSS total score and −1.3+6.3 points for the sleep/fatigue score were statistically significant versus baseline (both p<0.0001) [80]. Taken forward, the bedtime dosing of opicapone and the inferred efficacy against sleep/fatigue symptoms suggest that more work to understand which aspects of sleep might be improved with opicapone merits attention. For example, the inventors believe that optimization of the pharmacokinetic and pharmacodynamic profile of levodopa with opicapone is more likely to improve nighttime disabilities than sleep architecture. The OpicApone Sleep dISorder (OASIS) study (EudraCT number 2020-001176-15) is an open-label, single-arm, pilot study is designed to evaluate the effect of opicapone 50 mg on PD patients with end-of-dose motor fluctuations and a Parkinson's Disease Sleep Scale (PDSS-2) of ≥18. The primary endpoint is change from baseline to end of study in PDSS-2 Total scores, and secondary measures include the change from baseline in Parkinson's Disease Fatigue Scale (PFS-16) and the change from baseline in Domain K (sleep and wakefulness) of the Movement Disorder Society-sponsored Non-motor Rating Scale (MDS-NMS) [110].

Finally, pain, one of the most common and troublesome non-motor symptoms of PD, is another non-motor symptom which is often known to correlate with the motor OFF-state and be dopa-responsive [111-113]. In particular, optimizing levodopa regimens may be advantageous in treating this symptom since levodopa (but not apomorphine) has been shown to normalize pain thresholds in PD patients. Obviously, any study examining the effects of an intervention on a particular non-motor symptom must ensure that the patient population is enriched with patients who experience that symptom. Therefore, in another randomized, double-blind, placebo-controlled, clinical study (the OpiCapone Effect on motor fluctuations and pAiN [OCEAN] study; EudraCT number 2020-001175-32) that is currently underway to evaluate the effect of opicapone 50 mg in PD patients with end-of-dose motor fluctuations and associated pain, eligible patients must not only have PD (Hoehn and Yahr stages I-III during ON), be on a stable levodopa regimen and experiencing at least 1.5 hours of OFF daily OFF time despite optimal therapy, but also be experiencing PD associated pain for at least 4 weeks prior to screening as defined by a score of ≥12 on the King's Parkinson's Disease Pain Scale (KPPS). The primary efficacy measure is change from baseline in Domain 3 (fluctuation-related pain) of the KPPS, and secondary efficacy measures will also assess anxiety and depression as well as sleep and wakefulness among other measures [114].

7. Conclusions

Improving the efficacy of levodopa through the inhibition of peripheral COMT activity is an accepted option for the treatment of wearing-off in later stage PD. This has been comprehensively demonstrated in the clinical evaluation of entacapone, tolcapone and more recently, opicapone. While the significance of COMT in the periphery as a limiting factor in the perilous journey of levodopa to the brain is not contested, the present inventors here provide a good pharmacologic rationale to believe that earlier, effective COMT inhibition will bring clinical benefits in the management of long-term disease.

Some of the reasons for the current late positioning of COMT inhibitors in PD algorithms are historical, in that the DDCIs were developed in an era when levodopa was first being introduced into therapy when there were no barriers on the stage of illness where it could be used—and there were no alternatives. Other reasons relate to the process of drug development and regulatory approval for symptomatic treatments for PD. The standard practice has been to first trial drugs in an abundant population of later stage patients with wearing-off where improvements in ON time became a standardized end point accepted by regulatory authorities that then brought a product to the market and generated income. As a consequence, the late arrival of COMT inhibitors resulted in them being pigeonholed for this more advanced treatment group.

Another reason is a fundamental lack of basic understanding of the metabolism of levodopa and the importance of COMT as a limiting factor in the availability of the drug to the brain. In particular, the shunting of levodopa metabolism to the COMT pathway when used with a DDCI. This is probably because levodopa has been around for more than 60 years and its efficacy is unchallenged so there is little reason for physicians to worry about how it works and how peripheral metabolism of the drug influences clinical outcome.

The use of COMT inhibitors in all stages of PD was not thought feasible until now because of the constraints of earlier generations of COMT inhibitors. The explanation of the FIRST-STEP and STRIDE-PD studies above shows why the short duration of effect of entacapone was a critical limitation in early use. In contrast, the inventors believe that there is now the potential for the expansion of COMT inhibitor use to be fully explored based on the once daily administration of opicapone, its long duration of effect and its proven clinical efficacy in the classical later stage indication. The inventors proposed the early use based on their analysis of the basic science surrounding opicapone and the relevance to the metabolism of levodopa. As explained in more detail below, this hypothesis is supported by the appropriate clinical evaluation in an early patient population with outcome measures that demonstrate opicapone improves motor function, delays the onset of motor complications and treats some non-motor symptoms of PD.

### BRIEF DESCRIPTION OF THE DRAWINGS

The invention will now be described in detail with reference to the accompanying drawings, in which:

FIG. 1 shows the primary endpoint at the end of the double-blind phase: the change from the double-blind baseline to the end of the double-blind period in Movement Disorder Society-Unified Parkinson's Disease Rating Scale (MDS-UPDRS) Part III total score compared to placebo. FIG. 1 demonstrates a symptomatic reduction of 2.2 points (p=0.010).

FIG. 2 shows the longitudinal data confirming the magnitude of the effect in Movement Disorder Society-Unified Parkinson's Disease Rating Scale (MDS-UPDRS) Part III total score, compared to placebo, increased over time. DB=Double-Blind; LS=Least Square; SE=Standard Error; p=p value; *=statistically significant.

FIGS. 3A and 3B show the change from the double-blind baseline to the end of the double-blind period in MDS-UPDRS Part II+III total score compared to placebo. FIG. 3A demonstrates a symptomatic reduction of 2.8 points (p=0.036). FIG. 3B shows the longitudinal data confirming the magnitude of the effect, compared to placebo, increased over time. DB=Double-Blind; LS=Least Square; SE=Standard Error; p=p value; *=statistically significant.

FIGS. 4A and 4B show the proportion of opicapone-treated patients with an improvement in their clinical condition compared to placebo-treated patients. FIG. 4A shows the proportion of opicapone-treated patients that reported an improvement in their clinical condition (PGI-I score) compared to placebo-treated patients (p=0.026). FIG. 4B shows the proportion of opicapone-treated patients with an improvement in their clinical condition when assessed by a clinician (the CGI-I score) compared to placebo-treated patients (p=0.493).

FIG. 5 shows the improvement compared to placebo-treated patients in the PDSS-2 (p=0.039) with no worsening observed in the opicapone-treated group. DB=Double-Blind; LS=Least Square; SE=Standard Error; p=p value.

FIGS. 6A and 6B show the improvement compared to placebo-treated patients in the MDS-UPDRS Part II total score (p=0.120) with no worsening observed in the opicapone-treated group. FIG. 6A shows a positive trend towards a therapeutic effect was seen for the MDS-UPDRS Part II total score. FIG. 6B shows the magnitude of the difference increasing over time. DB=Double-Blind; LS=Least Square; SE=Standard Error; p=p value.

FIG. 7 shows a positive trend towards a therapeutic effect was also seen for the NMSS (p=0.102). DB=Double-Blind; LS=Least Square; SE=Standard Error; p=p value.

FIG. 8 shows a lower proportion of opicapone-treated patients reported motor complications (5.5%) compared to placebo-treated patients (9.8%).

FIG. 9 shows the design of a clinical study including timelines and approximate visit dates. EOS=End-of-Study Visit; DDCI=DOPA decarboxylase inhibitor; L-DOPA=levodopa; PSV=Post-Study Visit; QD=once daily.

FIG. 10 shows the distribution of patients during the clinical trial including randomization of 355 subjects. 322 subjects completed the double-blind phase. OPC=opicapone; FAS=Full Analysis Set; PP=Per Protocol.

### DETAILED DESCRIPTION OF THE INVENTION

A. Definitions

The following definitions apply to the terms used throughout this specification, unless otherwise limited in specific instances.

The term “idiopathic Parkinson's disease” encompasses most (80-85%) Parkinson's disease (diagnosed according to either the United Kingdom Parkinson's Disease Society Brain Bank Clinical Diagnostic Criteria or the Movement Disorder Society criteria) and excludes atypical parkinsonism, secondary [acquired or symptomatic] parkinsonism and Parkinson-plus syndrome, for example, drug-induced parkinsonism, vascular parkinsonism, normal pressure hydrocephalus, corticobasal degeneration, progressive supranuclear palsy and multiple system atrophy. It typically involves prominent bradykinesia and variable associated extrapyramidal signs and symptoms. It is typically accompanied by degeneration of the nigrostriatal dopaminergic system, with neuronal loss and reactive gliosis in the substantia nigra found at autopsy. In idiopathic Parkinson's disease, α-synuclein typically accumulates in neuronal perikarya (Lewy bodies) and neuronal processes (Lewy neurites).

The term “early idiopathic Parkinson's disease” or “early Parkinson's disease” refers to the early stage of the disease, when overt symptoms allow a diagnosis of idiopathic Parkinson's disease (according to either the United Kingdom Parkinson's Disease Society Brain Bank Clinical Diagnostic Criteria or the Movement Disorder Society criteria) but a complete response to treatment is possible without the presence of motor complications, such as motor fluctuations and/or dyskinesia. In particular, this patient group's Parkinson's disease is treatable (i.e. their symptoms can be controlled) with preparations of levodopa and a DDCI. As discussed below, this patient population exhibits a low total score of MDS-UPDRS Part IV A+B+C (e.g., zero) and/or a low number of positive symptoms in the 9-items Wearing off Questionnaire (WOQ-9) (e.g., below 2, preferably zero).

The term “symptoms of Parkinson's disease”includes both motor symptoms (e.g. tremor, rigidity, bradykinesia and postural instability) and non-motor symptoms (e.g. cognitive changes, gastrointestinal symptoms, loss of sight, taste and/or smell, pain, fatigue, light-headedness, sexual problems, sleep disorders and weight loss). Such symptoms can be assessed using one or more of the symptomatic readouts described below.

The term “motor complications” relates to Parkinson's disease symptoms which are related to levodopa therapy. They arise when levodopa/DDCI therapy alone no longer provides complete control of the patient's symptoms. They include motor fluctuations and/or dyskinesia. Motor complications are sustained, but not necessarily regular or predictable, such that they quantifiably and negatively impact on the patient's quality of life (QoL). “Clinically diagnosed motor complications” generally result in a total score of MDS-UPDRS Part IV A+B+C greater than 6, preferably greater than 3, more preferably greater than 0 and/or one or more positive symptoms in the 9-items Wearing off Questionnaire (WOQ-9). A total score of MDS-UPDRS Part IV A+B+C greater than 0 (zero) is the most preferred definition of clinically diagnosed motor complications. It should be noted that motor complications can be the same as motor symptoms of Parkinson's disease. However, a motor symptom which is initially treatable by levodopa/DDCI therapy, but which re-emerges at a later stage of disease in spite of maintaining levodopa/DDCI therapy, is then considered a motor complication.

The term “motor fluctuations” includes end-of-dose fluctuations (also known as the wearing-off phenomenon), paradoxical fluctuations and unpredictable on/off periods.

The term “off period” or “off episode” is defined as the times during which a patient treated with levodopa no longer experiences its symptomatic benefit and is said to be in an “off” state. On the other hand, when a patient treated with levodopa experiences its symptomatic benefit the patient is said to be in an “on” state.

The term “end-of-dose motor fluctuations”, also known as the “wearing off” phenomenon, relates to the predictable re-emergence or worsening of symptoms before administration of the next dose of levodopa/DDCI therapy. Typically, such re-emergence or worsening of symptoms starts 3-4 hours after a dose of levodopa, as the medication wears off and symptoms re-emerge or worsen. Symptoms then typically improve 15-45 minutes after the next levodopa dose is taken.

The term “dyskinesia” or “levodopa-induced dyskinesia” includes peak dose dyskinesia, diphasic dyskinesia and off dyskinesia. Common symptoms include chorea and dystonia. Less common symptoms include akathasia (excessive motor restlessness), a high stepped overshooting gait, rapid alternating movements (RAM) of legs, blepharospasm, and mixed pattern of abnormal movements (Fahn S., Ann. Neurol., 2000, 47, S2-S9).

The term “adjunctive therapy”, also known as “adjunct therapy”, “add-on therapy”, or “adjuvant care”, is therapy that is given in addition to the primary or initial therapy to maximize its effectiveness. In the current application, levodopa is the primary therapy and the DCCI and COMT inhibitor (i.e. opicapone) are the adjunctive therapies.

The term “treatment-emergent adverse event” is defined as any event not present before exposure to the study drug or any event already present that worsens in either intensity or frequency after first intake of study drug until 2 weeks after last intake of the study drug.

Other variations to the disclosed embodiments can be understood and effected by those skilled in the art in practicing the claimed invention, from a study of the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage.

B. Treatment of Early Parkinson's Disease with L-DOPA/DDCI and Opicapone

The invention provides opicapone for use as adjunctive therapy to preparations of levodopa and a DOPA decarboxylase inhibitor (DDCI) in the treatment of early Parkinson's disease; characterised in that the patient with early Parkinson's disease is treatable with preparations of levodopa and a DDCI without clinically diagnosed motor complications (either any type of motor fluctuations and/or dyskinesia).

The invention also provides the use of opicapone in the manufacture of a medicament for use as adjunctive therapy to preparations of levodopa and a DDCI in the treatment of early Parkinson's disease; characterised in that a patient with early Parkinson's disease is treatable with preparations of levodopa and a DDCI without clinically diagnosed motor complications (either any type of motor fluctuations and/or dyskinesia).

The invention also provides a method of treating early Parkinson's disease comprising administering opicapone as adjunctive therapy to preparations of levodopa and a DDCI to a patient in need thereof; characterised in that the patient with early Parkinson's disease is treatable with preparations of levodopa and a DDCI without clinically diagnosed motor complications (either any type of motor fluctuations and/or dyskinesia).

Symptoms and their Treatment

An important aspect of the invention is that it improves the treatment of symptoms characteristic of early stage Parkinson's disease, in patients wherein those symptoms are treatable with preparations of levodopa and a DDCI without motor complications. However, it is important to remember that a particular problem can present itself both as a symptom of early Parkinson's disease and as a motor complication at a later stage. For example, perhaps the most stereotypical problem in Parkinson's disease is a tremor.

A tremor is a common symptom of early Parkinson's disease and might initially be completely treatable by levodopa/DDCI treatment. In this case, the tremor is not a symptom whose treatment can be improved by the adjunctive opicapone therapy of the present invention (because it is already completely treated by levodopa/DDCI treatment). Alternatively, the tremor might initially be only partially treatable by levodopa/DDCI treatment. In this case, the tremor is a symptom whose treatment can be improved by the adjunctive opicapone therapy of the present invention. Importantly, the presence of this type of tremor does not mean that the patient suffers from motor complications (as defined herein). In summary, a symptom that is treatable by the adjunctive opicapone therapy of the present invention is one which is present during early Parkinson's disease but for which levodopa/DDCI treatment is not completely effective.

A tremor can also be a motor complication and might emerge or develop at a later stage of disease, for example as an end-of-dose motor fluctuation. In this case, the presence of the tremor might result in a clinical diagnosis of motor complications (as defined herein).

Methods for differentiating symptoms of early Parkinson's disease, which are present to some extent throughout the period when levodopa/DDCI treatment is effective, from motor complications, which emerge or develop after extended levodopa/DDCI treatment, are known to the skilled person and are described in more detail below.

The use of opicapone as an adjunctive therapy to preparations of levodopa and a DDCI in the treatment of early Parkinson's disease results in an improvement in one or more symptoms in the patient. In a preferred embodiment, the opicapone treatment results in an improvement in one or more symptoms in the patient compared to those which would be exhibited by a patient treated for the same period with preparations of levodopa and a DDCI without opicapone. This means that the rate at which the patient declines is reduced, stopped or reversed. In a more preferred embodiment, the use of opicapone results in an improvement in one or more symptoms in the patient compared to the same patient prior to initiating opicapone treatment. This means that the rate at which the patient declines is reversed. In an even more preferred embodiment, the opicapone results in a rapid improvement in one or more symptoms in the patient compared to the same patient prior to initiating opicapone treatment, for example, 24 weeks, preferably 12 weeks, more preferably 4 weeks and most preferably 2 weeks after treatment is initiated.

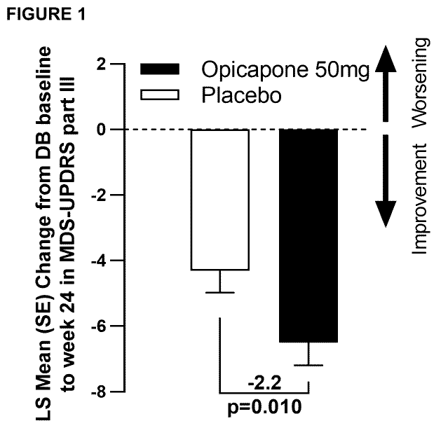

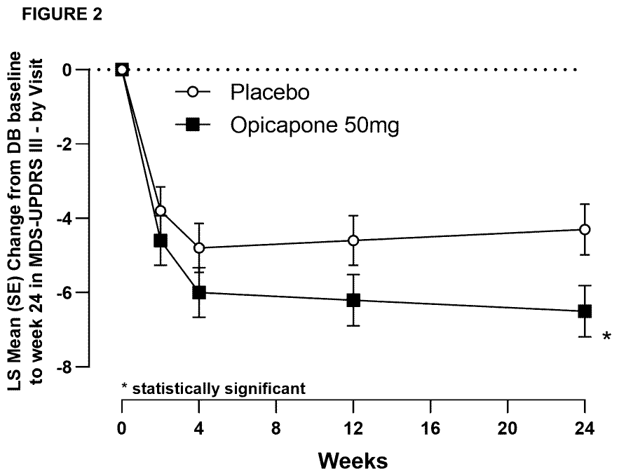

The exemplified trial described below assessed as a primary endpoint at the end of the double-blind phase, the change from the double-blind baseline to the end of the double-blind period in Movement Disorder Society-Unified Parkinson's Disease Rating Scale (MDS-UPDRS) Part III total score compared to placebo. The results shown in FIG. 1 demonstrate a symptomatic reduction of 2.2 points (p=0.010). Therefore, in a preferred embodiment, the use of opicapone in accordance with the present invention results in an improvement in the patient's score for one or more measures from the MDS-UPDRS Part III (Motor Examination), either compared to the score which would be achieved by a patient treated for the same period without opicapone, or compared to the score of the same patient prior to initiating opicapone treatment. Furthermore, in a highly preferred embodiment, the use of opicapone in accordance with the present invention results in an improvement in the MDS-UPDRS Part III (Motor Examination) total score of a patient treated according to the invention compared to the score which would be achieved by a patient treated for the same period without opicapone. In a particularly highly preferred embodiment, the use of opicapone in accordance with the present invention results in an improvement in the MDS-UPDRS Part III (Motor Examination) total score of a patient treated according to the invention compared to the score of the same patient prior to initiating opicapone treatment. FIG. 2 shows the longitudinal data confirms the effect, compared to placebo, is present from the earliest time points, but increases over time. The improvement is assessed by comparing the score of a patient prior to initiating opicapone treatment (baseline) with their score once the effect of opicapone has stabilised, for example, 24 weeks, preferably 12 weeks, more preferably 4 weeks and most preferably 2 weeks after treatment is initiated.

The MDS-UPDRS Part III (Motor Examination) total score assesses a number of symptoms, so within the preferred embodiments of the preceding paragraph, the use of opicapone in accordance with the present invention results in an improvement in the score for one or more measures selected from the group consisting of: speech; facial expression; rigidity; finger tapping; hand movement; pronation-supination movements of hands; toe tapping; leg agility; arising from chair; gait; freezing of gait; postural stability; posture; bradykinesia; postural tremor of the hand; kinetic tremor of the hands; rest tremor amplitude; and constancy of rest tremor; in a patient treated according to the invention compared to the score which would be achieved by a patient receiving placebo, and preferably compared to the score of the same patient prior to initiating opicapone treatment.

Other methods of assessing an improvement in the symptomatic treatment of early Parkinson's disease are known to the skilled person and are described in Section D below.

The exemplified trial described below assessed as secondary endpoints throughout the double-blind phase, the changes in (i) MDS-UPDRS scores: Parts I, II, III and IV, and Part II +III total; (ii) the modified Hoehn & Yahr staging total score during maximum ‘ON’ response; (iii) the Schwab and England scale score, (iv) the Parkinson's Disease Sleep Scale 2 (PDSS-2) total score; (v) the Movement Disorder Society-Non-Motor Symptom Scale (MDS-NMSS) total and subdomain scores; (vi) the Parkinson's Disease Questionnaire (PDQ-39) total and subdomain scores; and (vii) the Wearing off Questionnaire (WOQ-9) total and sub-section (motor and non-motor) scores. It also assesses the Clinical Global Impression of Improvement (CGI-I) and/or Patient's Global Impression of Improvement (PGI-I). Assessment through the open-label phase is ongoing.

Therefore, in a number of preferred embodiments, the opicapone treatment of the present invention results in an improvement in the patient compared to the score of the same patient prior to initiating opicapone treatment, in one or more of:
(i) the MDS-UPDRS Part I total score, in particular one or more of the group selected from:
   cognitive impairment; hallucinations and psychosis; depressed mood; anxious mood; apathy; features of dopamine dysregulation syndrome; sleep problems; daytime sleepiness; pain and other sensations; urinary problems; constipation problems; light headedness on standing; or fatigue;
(ii) the MDS-UPDRS Part II total score, in particular one or more of the group selected from:
   speech; saliva and drooling; chewing and swallowing; eating tasks; dressing; hygiene; handwriting; doing hobbies and other activities; turning in bed; tremor; getting out of bed, a car, or a deep chair; walking and balancing; or freezing;
(iii) the MDS-UPDRS Part II+III total score;
(iv) the modified Hoehn & Yahr staging total score during maximum ‘ON’ response;
(v) the Schwab and England scale score;
(vi) the PDSS-2 total score;
(vii) the MDS-NMSS total and subdomain scores;
(viii) the PDQ-39 total and subdomain scores;
(ix) the CGI-I score; and
(x) the PGI-I score.

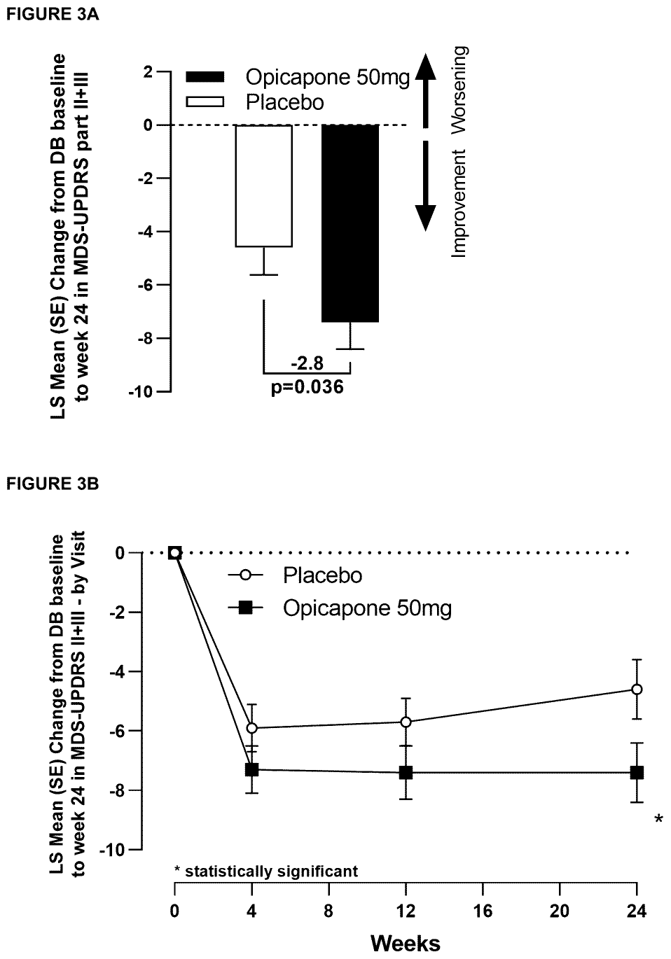

In particular, the results shown in FIG. 3 demonstrate a symptomatic reduction of 2.8 points (p=0.036) in the MDS-UPDRS Part II+III total score.

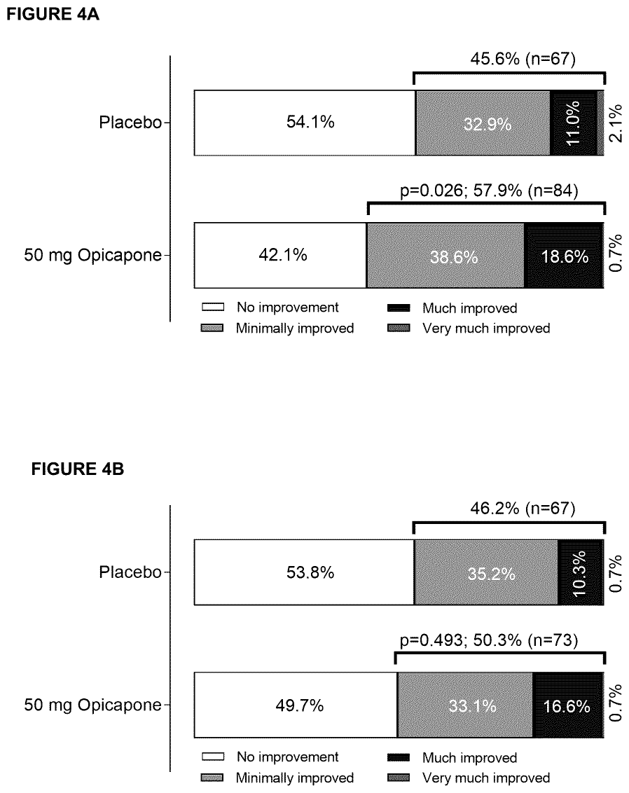

Furthermore, the results shown in FIG. 4a demonstrate a significantly higher proportion of opicapone-treated patients reported an improvement in their clinical condition (PGI-I score) with 57.9% showing an improvement compared to 45.6% of placebo-treated patients (p=0.026). A similar trend is shown in FIG. 4b when the clinical condition was assessed by a clinician (the CGI-I score) with 50.3% showing an improvement compared to 46.2% of placebo-treated patients (p=0.493).

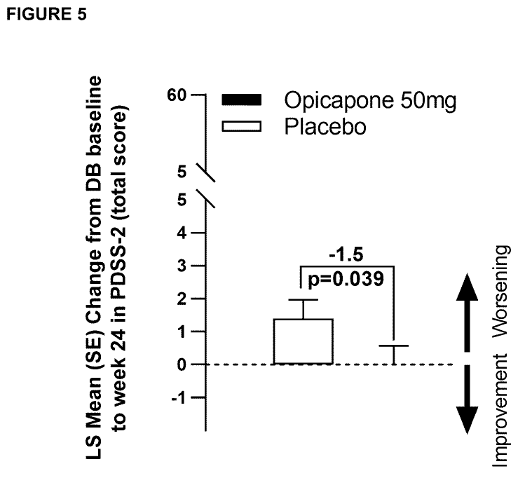

Additionally, the results shown in FIG. 5 demonstrate a significant improvement compared to placebo-treated patients in the PDSS-2 (p=0.039) with no worsening observed in the opicapone-treated group.

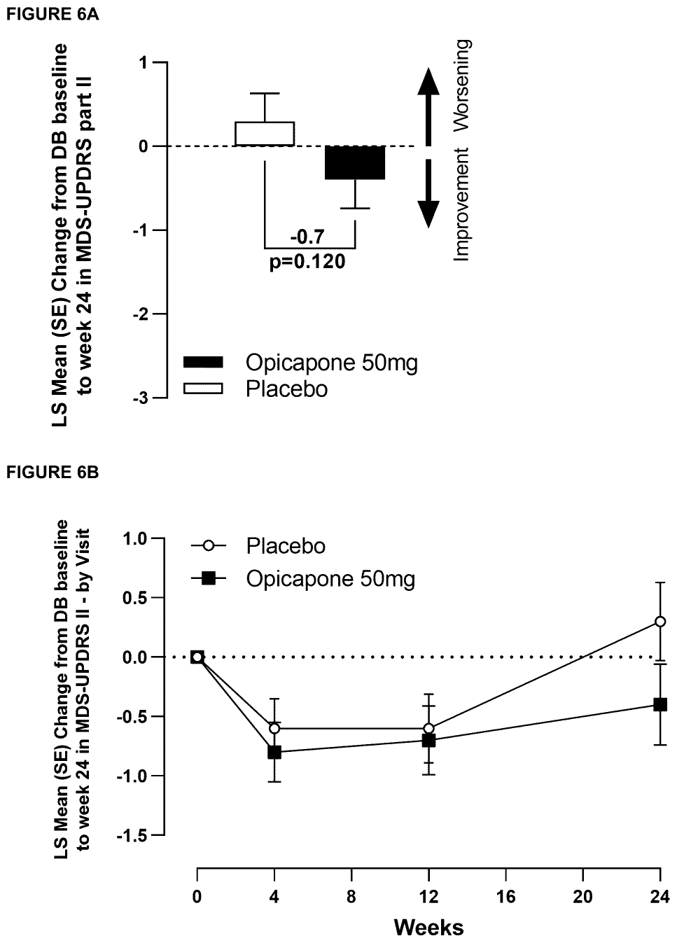

A positive trend towards a therapeutic effect was seen for the MDS-UPDRS Part II total score (FIG. 6a) with the magnitude of the difference increasing over time (FIG. 6b). This suggests larger group sizes or longer treatments might achieve statistical significance for other symptoms.

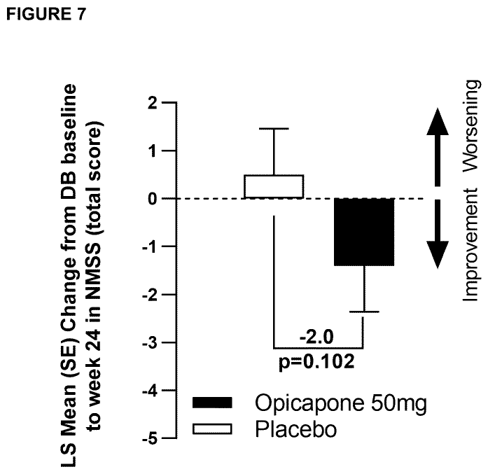

A positive trend towards a therapeutic effect was also seen for the NMSS (FIG. 7). Similar results were observed in the total scores in the subdomains of the NMSS scale except for Domain 7 (urinary). Significant improvements in urinary subdomain were observed in the opicapone treated group over placebo, especially at Week 12 and Week 24 (p<0.05).

No significant effects were observed for the MDS-UPDRS Part I, MDS-UPDRS Part IV or PDQ-39 total scores. However, the magnitude of the changes in both groups (opicapone and placebo) were less than 0.4 points for these symptoms. Therefore, longer treatment periods or larger trials may be required to observe significant effects in these symptoms.

In summary, the data demonstrate a statistically significant improvement in the primary endpoint and several secondary endpoints, as well as trends towards significance in other secondary endpoints where the effect was large enough to be observed.

Therefore, the data confirm the utility of opicapone as adjunctive therapy to preparations of levodopa and a DDCI in the treatment of (motor) signs and symptoms of Parkinson's disease; characterised in that the patient with Parkinson's disease is treatable with preparations of levodopa and a DDCI without clinically diagnosed motor complications.

Alternatively, the data confirm the utility of opicapone as adjunctive therapy to preparations of levodopa and a DDCI in the treatment of a patient with Parkinson's disease and insufficient control of (motor) signs and symptoms; characterised in that the patient with Parkinson's disease is treatable with preparations of levodopa and a DDCI without clinically diagnosed motor complications.

Furthermore, the data combine with the fact that opicapone is known to treat patients with Parkinson's disease already experiencing “end-of-dose motor fluctuations” (European label) or ““off” episodes” (US label) to confirm the utility of opicapone in treating Parkinson's disease without the need to assess or diagnose end-of-dose motor fluctuations. As such, the patient only needs to be diagnosed with a standard clinical diagnosis of bradykinesia and at least one of the following: resting tremor, muscular rigidity and postural reflex impairment (core symptoms). Furthermore, the data support opicapone for use in treating the symptoms and signs of Parkinson's disease in combination with levodopa, over the course of the disease (when the effect of levodopa wears off or becomes inconsistent and fluctuations in the therapeutic effect occur (“end of dose” or “on-off” type fluctuations)).

In one alternative embodiment, the use of opicapone in accordance with the present invention results in an improvement in the patient's score for one or more measures from the MDS-UPDRS Part I (non-motor aspects of experiences of daily living), either compared to the score which would be achieved by a patient treated for the same period without opicapone, or compared to the score of the same patient prior to initiating opicapone treatment. More preferably, the use of opicapone in accordance with the present invention results in an improvement in the MDS-UPDRS Part I total score of the patient compared to the score which would be achieved by a patient receiving placebo, and preferably compared to the score of the same patient prior to initiating opicapone treatment. In preferred examples of these embodiments, the use of opicapone results in an improvement in one or more measures selected from the group consisting of: cognitive impairment; hallucinations; psychosis; depressed mood; anxious mood; apathy; features of dopamine dysregulation syndrome; sleep problems; daytime sleepiness; pain; urinary problems; constipation problems; light headedness on standing; and fatigue; in a patient treated according to the invention compared to the score which would be achieved by a patient receiving placebo, and preferably compared to the score of the same patient prior to initiating opicapone treatment.

In a second alternative embodiment, the use of opicapone in accordance with the present invention results in an improvement in the patient's score for one or more measures from the MDS-UPDRS Part II (motor aspects of experiences of daily living), either compared to the score which would be achieved by a patient treated for the same period without opicapone, or compared to the score of the same patient prior to initiating opicapone treatment. More preferably, the use of opicapone in accordance with the present invention results in an improvement in the MDS-UPDRS Part II total score of the patient compared to the score which would be achieved by a patient receiving placebo, and preferably compared to the score of the same patient prior to initiating opicapone treatment. In preferred examples of these embodiments, the opicapone results in an improvement in one or more measures selected from the group consisting of: speech; salivation; drooling; chewing; swallowing; eating tasks; dressing; hygiene; handwriting; doing hobbies; turning in bed; tremor; getting out of bed, a car, or a deep chair; walking; balancing; and freezing; in a patient treated according to the invention compared to the score which would be achieved by a patient receiving placebo, and preferably compared to the score of the same patient prior to initiating opicapone treatment.

In a third preferred embodiment, the use of opicapone in accordance with the present invention results in an improvement in modified Hoehn & Yahr staging total score in a patient treated according to the invention compared to the score which would be achieved by a patient receiving placebo, and preferably compared to the score of the same patient prior to initiating opicapone treatment.

In a fourth preferred embodiment, the use of opicapone in accordance with the present invention results in an improvement in Schwab and England scale score in a patient treated according to the invention compared to the score which would be achieved by a patient receiving placebo, and preferably compared to the score of the same patient prior to initiating opicapone treatment.

In a fifth preferred embodiment, the use of opicapone in accordance with the present invention results in an improvement in PDSS-2 total score in a patient treated according to the invention compared to the score which would be achieved by a patient receiving placebo, and preferably compared to the score of the same patient prior to initiating opicapone treatment.

In a sixth preferred embodiment, the use of opicapone in accordance with the present invention results in an improvement in the MDS-NMSS total and subdomain scores in a patient treated according to the invention compared to the score which would be achieved by a patient receiving placebo, and preferably compared to the score of the same patient prior to initiating opicapone treatment. In a preferred example of this embodiment, the opicapone results in an improvement in one or more sub-domains selected from the group consisting of: cardiovascular; sleep; fatigue; mood; cognition; perceptual problems; attention; memory; gastrointestinal; urinary; and sexual function; in a patient treated according to the invention compared to the score which would be achieved by a patient receiving placebo, and preferably compared to the score of the same patient prior to initiating opicapone treatment.

In a seventh preferred embodiment, the use of opicapone in accordance with the present invention results in an improvement in the PDQ-39 total and subdomain scores in a patient treated according to the invention compared to the score which would be achieved by a patient receiving placebo, and preferably compared to the score of the same patient prior to initiating opicapone treatment. In a preferred example of this embodiment, the opicapone results in an improvement in one or more sub-domains selected from the group consisting of: mobility; activities of daily living (ADL); emotions; stigma; social support; cognitions; communication; and bodily discomfort; in a patient treated according to the invention compared to the score which would be achieved by a patient receiving placebo, and preferably compared to the score of the same patient prior to initiating opicapone treatment.

In an eighth preferred embodiment, the use of opicapone in accordance with the present invention results in an improvement in the CGI-I score in a patient treated according to the invention compared to the score which would be achieved by a patient receiving placebo, and preferably compared to the score of the same patient prior to initiating opicapone treatment.

In a ninth preferred embodiment, the use of opicapone in accordance with the present invention results in an improvement in the PGI-I score in a patient treated according to the invention compared to the score which would be achieved by a patient receiving placebo, and preferably compared to the score of the same patient prior to initiating opicapone treatment.

Whilst the improvements are generally assessed by comparing the score of the patient prior to initiating opicapone treatment (baseline) with their score at the end of the double-blind and/or open-label periods, the trial assesses symptoms at multiple points (visits) throughout the trial, so the improvement can be assessed at any time once the effect of opicapone has stabilised. For example, the improvement may be assessed 24 weeks, preferably 12 weeks, more preferably 4 weeks and most preferably 2 weeks, after treatment is initiated.

Shorter periods of time between initial and final assessment differentiate symptomatic treatments from any possible disease-modifying effect that is observed.

Motor Complications and Suppression of their Emergence

Although the patient population selected for treatment according to the present invention do not suffer from motor complications, the exemplified trial assessed the emergence of motor complications throughout the double-blind period. Assessment through the open-label phase is ongoing.

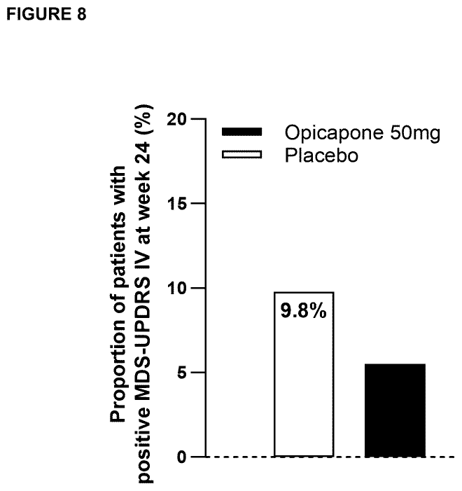

As shown in FIG. 8, a lower proportion of opicapone-treated patients reported motor complications (5.5%) compared to placebo-treated patients (9.8%).

Therefore, in a highly preferred embodiment, treatment according to the present invention suppresses the emergence of one or more motor complications during treatment with opicapone and in spite of maintaining levodopa/DDCI therapy.

The emergence of one or more motor complications during treatment according to the invention was assessed using the methods for assessing motor complications described in Section D, below.

In particular, within this highly preferred embodiment, the suppression of the emergence of motor complications during treatment according to the invention results in one or more of: maintaining two or fewer, preferably one or zero, more preferably zero positive symptoms in the WOQ-9 that improve after the next dose of levodopa; and/or maintaining an average daily off-time of less than 1.5 hours, preferably less than 1.0 hours, more preferably less than 0.5 hours, most preferably zero hours.

The trial described below assesses as a primary endpoint at the end of the open-label phase, the change from open-label baseline to the end of the open-label period in MDS-UPDRS Part IV (Motor Complications) total score. Therefore, in a highly preferred embodiment, the opicapone treatment of the present invention results in a reduction in the increase in the MDS-UPDRS Part IV total score of the patient compared to the increase which would be observed in the absence of opicapone treatment over the same period. Within this highly preferred embodiment, the opicapone treatment reduces the MDS-UPDRS Part IV total score of the patient to 80% or less, preferably 60% or less, more preferably 40% or less, even more preferably 20% or less, most preferably 10% or less, of the total score which would be observed in the absence of opicapone treatment over the same period.

### Stability of Levodopa/DDCI Therapy

During sustained levodopa/DDCI therapy, patients might need to increase the levodopa dose, for example, from three 100 mg levodopa doses per day (300 mg daily dose) to four 100 mg levodopa doses per day (400 mg daily dose). Therefore, in a highly preferred embodiment, treatment according to the present invention allows the patient to maintain a stable daily dose of levodopa/DDCI therapy for a period of at least 3 months, preferably at least 24 weeks, more preferably at least 1 year. More preferably, the treatment according to the present invention allows the patient to maintain a stable dose frequency of levodopa/DDCI therapy (e.g., more frequent smaller doses of levodopa/DDCI therapy throughout the day) for a period of at least 3 months, preferably at least 24 weeks, more preferably at least 1 year.

In a more preferred embodiment, treatment according to the present invention allows the patient to reduce the daily dose of L-DOPA/DDCI by extending the dosing intervals and/or by reducing the amount of L-DOPA/DDCI per dose. For example, the patient is able to reduce the number of daily dosages of levodopa/DDCI by one per day, preferably two per day, or more preferably three per day. For example, the dosage might be reduced from four 100 mg levodopa doses per day (400 mg daily dose) to three 100 mg levodopa doses per day (300 mg daily dose), preferably from five 100 mg levodopa doses per day (500 mg daily dose) to three 100 mg levodopa doses per day (300 mg daily dose).

### Patient Population

The treatment of early Parkinson's disease with levodopa/DDCI and opicapone is preferably directed to humans, more preferably adult humans, even more preferably adult humans who are at least 30 years old, preferably at least 50 years old, more preferably at least 65 years old.

The patient with Parkinson's disease preferably suffers from idiopathic Parkinson's disease. As the data confirm opicapone can treat patients with end-of-dose motor fluctuations and those yet to suffer from motor fluctuations, the patient only needs to be diagnosed with a standard clinical diagnosis of bradykinesia and at least one of the following: resting tremor, muscular rigidity and postural reflex impairment (core symptoms). Therefore, in one embodiment, the patient has not undergone assessment for end-of-dose motor fluctuations. In another embodiment, the patient with Parkinson's disease has insufficient control of (motor) signs and symptoms despite treatment with levodopa/DDCI.

The use of opicapone in addition to levodopa/DDCI further improves the treatment of one or more symptoms of idiopathic Parkinson's disease discussed above which are already partially treated when levodopa/DDCI therapy is initiated in a patient.

The patient suffers from early stage idiopathic Parkinson's disease so does not suffer from motor complications. Methods of assessing the presence or absence of motor complications are known to the skilled person. Section D, below, describes methods for assessing symptoms in Parkinson's disease including those that can assess motor complications.

The most widely used clinical scale for assessing the clinical status of patients with Parkinson's disease is the Unified Parkinson's Disease Rating Scale (UPDRS) (Fahn S, Elton R L, UPDRS Program Members. Unified Parkinson's disease rating scale. In Recent Developments in Parkinson's Disease, Vol. 2, eds Fahn S, Marsden C D, Goldstein M. Florham Park, NJ, USA: Macmillan Healthcare Information, 1987:153-63, 293-304). The primary measure to define if a patient suffers from early stage idiopathic Parkinson's disease is based on the total score of MDS-UPDRS Part IV A+B+C being less than 6, preferably less than 3, in particular ‘0’ (zero). MDS-UPDRS Part IV specifically evaluates motor complications of therapy.

Therefore, in the most preferred embodiment of the invention, the patient with Parkinson's disease treatable with preparations of levodopa and a DDCI without clinically diagnosed motor complications displays a total score of MDS-UPDRS Part IV A+B+C of zero when being treated with preparations of levodopa and a DDCI.

In an alternative preferred embodiment, the patient with Parkinson's disease treatable with preparations of levodopa and a DDCI without clinically diagnosed motor complications displays two or less, preferably one or less, more preferably zero positive symptoms in the WOQ-9 that improve after the next dose of levodopa.

In a more preferred version of the embodiment above, the motor complications absent in the patient with Parkinson's disease treatable with preparations of levodopa and a DDCI without clinically diagnosed motor complications are selected from the group consisting of tremor, mood changes, slowness in movement, reduced dexterity, stiffness, anxiety/panic attacks, cloudy mind/slowness, muscle cramp, and pain/aching. These are the motor complications listed in the WOQ-9. Most preferably, the motor complications absent in the patient with Parkinson's disease treatable with preparations of levodopa and a DDCI without clinically diagnosed motor complications are selected from the group consisting of tremor, anxiety/panic attacks and slowness of movement. These particular symptoms are best suited to capturing patients with motor complications, such as end-of-dose motor fluctuations (Stacy M. and Hauser R., J. Neural. Transm. 2007, 114, 211-217).

In another alternative preferred embodiment, the motor complications are selected from the group consisting of motor fluctuations and/or dyskinesia, as diagnosed by the skilled clinician.

In general, a patient suffering from early stage idiopathic Parkinson's disease will have been treated for Parkinson's disease for a shorter period than patients beyond the early stages of Parkinson's disease. In a preferred embodiment, the patient commenced treatment with levodopa within the past 5 years, preferably within the past 1 to 3 years, more preferably within the past 1 year, or even more preferably within the past 6 months, most preferably the patient is not previously treated with levodopa.

In general, a patient suffering from early stage idiopathic Parkinson's disease will have a modified Hoehn and Yahr stage lower than patients beyond the early stages of Parkinson's disease. In a preferred embodiment, the patient has a modified Hoehn and Yahr stage 1 to 3, preferably 1.0 to 2.5, more preferably 1.0 to 2.0 in the on state prior to treatment with opicapone.

In general, a patient suffering from early stage idiopathic Parkinson's disease will receive less levodopa per day than patients beyond the early stages of Parkinson's disease. In a preferred embodiment, the patient receives 600 mg or less of levodopa per day, preferably 500 mg or less per day, more preferably 400 mg or less per day and even more preferably 300 mg or less per day and most preferably less than 300 mg per day.

In general, a patient suffering from early stage idiopathic Parkinson's disease will receive fewer doses of levodopa per day than patients beyond the early stages of Parkinson's disease. In a preferred embodiment, the patient receives levodopa 6 times or less per day, preferably 5 times or less, more preferably 4 times or less, even more preferably 3 times or less.

In a particularly preferred embodiment, a patient suffering from early stage idiopathic Parkinson's disease will have received treatment with levodopa/DDCI (e.g., controlled-release, immediate-release or combined controlled immediate-release) for at least 1 year, and at a stable regimen with daily dose in the range 300 to 500 mg, 3 to 4 times a day, for at least 4 weeks prior to initiating opicapone.

In general, a patient suffering from early stage idiopathic Parkinson's disease will not previously have been treated with a COMT inhibitor. In a preferred embodiment, the patient is not currently treated with a COMT inhibitor, preferably the patient has never been treated with a COMT inhibitor.

In general, a patient suffering from early stage idiopathic Parkinson's disease and treated with levodopa will receive immediate-release levodopa because controlled-release levodopa provides no added benefit in relation to the immediate-release levodopa. In a preferred embodiment, the patient is not currently treated with controlled-release levodopa, more preferably the patient has never been treated with controlled-release levodopa.

Patients suffering from the very early stage idiopathic Parkinson's disease might never have been treated for Parkinson's disease using pharmaceutical interventions. In a preferred embodiment, the patient has never been treated for Parkinson's disease.

### Opicapone Dosage and Regimens

Opicapone is a long-acting COMT inhibitor compared to other known COMT inhibitors. In a preferred embodiment, the opicapone is administered once daily or once weekly, preferably once daily.

Opicapone is effective with low toxicity and displays good pharmacodynamics properties at relatively low doses. In a preferred embodiment, the unit dose of opicapone is 5 to 100 mg, preferably 25 to 75 mg, more preferably 25 or 50 mg, most preferably 50 mg.

Opicapone can interact with food. In a preferred embodiment, the opicapone is administered more than 1 hour before or after a meal.

Opicapone can interact with levodopa. In a preferred embodiment, the opicapone is administered more than 1 hour before or after administration of levodopa.

In a more preferred embodiment, the opicapone is administered at or near to bedtime, e.g, less than 1 hour before sleep or even less than 30 minutes before sleep.

Opicapone showed good tolerability and a low incidence of Adverse Events (AEs) including Treatment Emergent Adverse Events. Indeed, opicapone was found to be well tolerated in patients without clinically diagnosed motor complications with a more favourable safety profile than previous studies in patients with clinically diagnosed motor complications.

Opicapone-treated patients saw no increase in treatment emergent adverse events including nervous system disorders such as dyskinesia.

In a preferred embodiment, the treatment lasts at least 24 weeks, preferably at least 1 year.

In a preferred embodiment related to the previous embodiments, the administration of opicapone results in an improvement in one or more of the symptomatic readouts described above without inducing one or more motor complications described above.

In another preferred embodiment related to the previous embodiments, the administration of opicapone results in an improvement in one or more of the symptomatic readouts described above without inducing one or more treatment-emergent adverse events described above.

The embodiments and preferred embodiments described above apply equally for the use of opicapone in the manufacture of a medicament and the method of treating Parkinson's disease symptoms described at the top of Section B.

C. Clinical Protocols

### Clinical Trial Design

The applicant performed a Phase III study evaluating the efficacy and safety of opicapone (50 mg) in patients with early idiopathic Parkinson's disease receiving treatment with levodopa/DDCI, and who are without signs of any motor complication (e.g., fluctuations in the motor response and/or involuntary movements and/or dyskinesia).

Subjects were between 30 and 80 years of age, inclusive, diagnosed with early idiopathic Parkinson's disease according to the United Kingdom Parkinson's Disease Society Brain Bank Clinical Diagnostic Criteria within the previous 5 years, with disease severity stage 1-2.5 (according to modified Hoehn & Yahr staging) and a MDS-UPDRS Part III score>20. Alternatively, the subjects were diagnosed with early idiopathic Parkinson's disease according to the MDS Non-motor Symptoms Scale (MDS-NMSS) criteria within the previous 5 years, with disease severity stage 1-2.5 (according to modified Hoehn & Yahr staging) and a MDS-UPDRS Part III score>20. Subjects received treatment with levodopa/DDCI at a stable regimen for at least 4 weeks prior to randomisation, had no signs of motor complications (consisting of fluctuations in the motor response and/or involuntary movements or dyskinesias), and were naive to COMT inhibitors.

### Inclusion Criteria

Subjects were eligible to be included in the study only if all the following criteria applied:
1. Capable of giving signed informed consent.
2. Subjects must be 30 to 80 years of age, inclusive, at the time of signing the informed consent form for the double-blind trial.
3. Diagnosed with idiopathic Parkinson's disease according to the UK Parkinson's Disease Society Brain Bank Clinical Diagnostic Criteria within the previous 5 years and/or MDS Non-motor Symptoms Scale (MDS-NMSS) within the previous 5 years.
4. Disease severity stages 1-2.5 (according to the modified Hoehn & Yahr staging)
5. Signs of treatable motor disability for a minimum of 4 weeks before screening, with minimum threshold with MDS-UPDRS Part III score of ≥20 at both screening and Visit 2, despite stable anti-Parkinson's disease therapy.
6. Receiving treatment with L-DOPA/DDCI (either controlled-release, immediate-release or combined controlled immediate-release) for at least 1 year, and at a stable regimen for at least 4 weeks prior to Visit 2 at a daily dose in the range 300 to 500 mg, 3 to 4 times a day.
7. Naive to COMT inhibitors (including opicapone).
8. Male or female.
   A male subject must agree to use contraception of this protocol during the treatment period and until the PSV, and refrain from donating sperm during this period.
   A female subject is eligible to participate if she is not pregnant, not breastfeeding, and at least 1 of the following conditions applies:
(i) Not a woman of childbearing potential (WOCBP).

OR
(ii) A WOCBP who agrees to follow the contraceptive guidance during the treatment period and until the PSV.
9. Results of the screening laboratory tests are considered clinically acceptable by the Investigator (i.e., not clinically relevant for the well-being of the subject or for the purpose of the study).

### Exclusion Criteria

Subjects were excluded from the study if any of the following criteria applied:
1. Non-idiopathic PD (for example, atypical parkinsonism, secondary [acquired or symptomatic] parkinsonism, Parkinson-plus syndrome).
2. Signs of motor complications with a total score of MDS-UPDRS Part IV A+B+C greater than ‘0’ (zero).
3. Treatment with prohibited medication: COMT inhibitors (e.g., entacapone, tolcapone), antiemetics with antidopaminergic action (except domperidone) or Duopa™ (carbidopa/levodopa intestinal gel) within the 4 weeks before screening.
4. Concomitant use of monoamine oxidase (MAO-A and MAO-B) inhibitors (e.g. phenelzine, tranylcypromine and moclobemide) other than those for the treatment of PD.
5. Previous or planned (during the entire study duration) deep brain stimulation.
6. Previous stereotactic surgery (eg, pallidotomy, thalamotomy) for Parkinson's disease or with planned stereotactic surgery during the study period.
7. Any investigational medicinal product within the 3 months (or within 5 half-lives, whichever is longer) before screening.
8. Any medical condition that might place the subject at increased risk or interfere with study assessments.
9. Past (within the past year) or present history of suicidal ideation or suicide attempts, as determined by a positive response (‘Yes’) to either Question 4 or Question 5 on the suicidal ideation portion of the Columbia-Suicide Severity Rating Scale (C-SSRS) (Screening questions).
10. Current or previous (within the past year) diagnosis of psychosis, severe major depression, or other psychiatric disorders that, based on the Investigator's judgment, might place the subject at increased risk or interfere with assessments.
11. A clinically relevant electrocardiogram (ECG) abnormality (relevance should be assessed by a cardiologist if needed).
12. Current evidence of unstable cardiovascular disease, including but not limited to uncontrolled hypertension, myocardial infarction with important systolic or diastolic dysfunction, unstable angina, congestive heart failure (New York Heart Association Class ≥III), and significant cardiac arrhythmia (Mobitz II 2^{nd} or 3^{rd} degree AV block or any other arrhythmia causing hemodynamic repercussions as symptomatic bradycardia or syncope).
13. Prior renal transplant or current renal dialysis.
14. Pheochromocytoma, paraganglioma or other catecholamine secretive neoplasm.
15. Known hypersensitivity to any ingredients of the study treatment.
16. History of neuroleptic malignant syndrome (NMS) or NMS-like syndromes, or non-traumatic rhabdomyolysis.
17. Malignancy within the past 5 years (eg, melanoma, prostate cancer), excluding cutaneous basal or squamous cell cancer resolved by excision.
18. Unstable active narrow-angle or unstable wide-angle glaucoma.
19. History of or current evidence of any relevant disease in the context of this study, ie, with respect to the safety of the subject or related to the study conditions, eg, which may influence the absorption or metabolism (such as a relevant liver disease) of the study treatment.
20. Any abnormality in the liver enzymes (alanine aminotransferase [ALT] and/or aspartate aminotransferase [AST]) >2 times the upper limit of the normal range, in the screening laboratory tests results.
21. Plasma sodium less than 130 mmol/L, white blood cell count less than 3000 cells/mm³, or any other relevant clinical laboratory abnormality that, in the Investigator's opinion, may compromise the subject's safety.
22. Evidence of an impulse control disorder (ICD) (one or more positive modules on the modified Minnesota Impulsive Disorders Interview (mMIDI)). A module is considered positive if a subject gives a positive answer (rarely=1, occasionally=2, frequently=3) to any question after answering in the affirmative to a gateway (initial) question.

### Randomisation

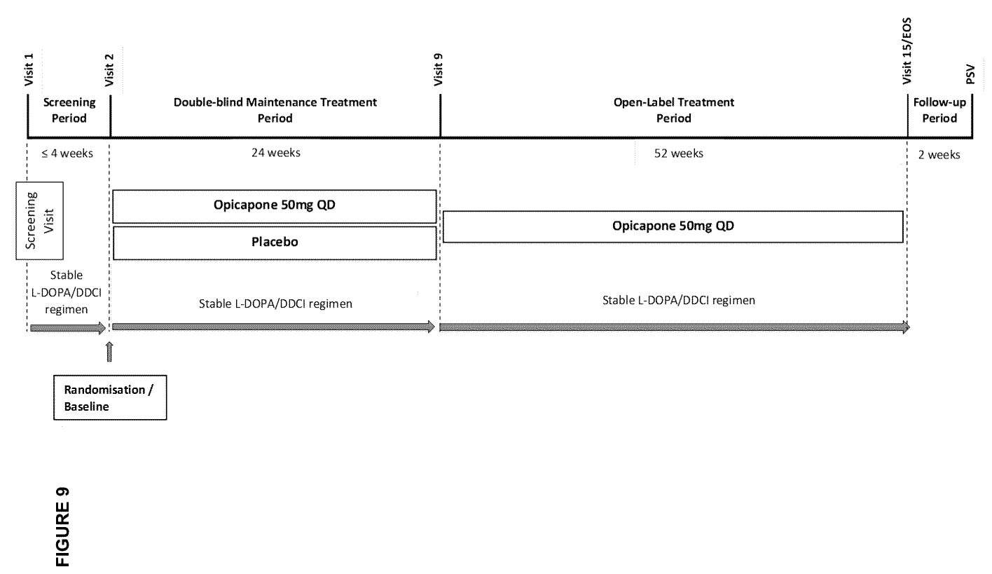

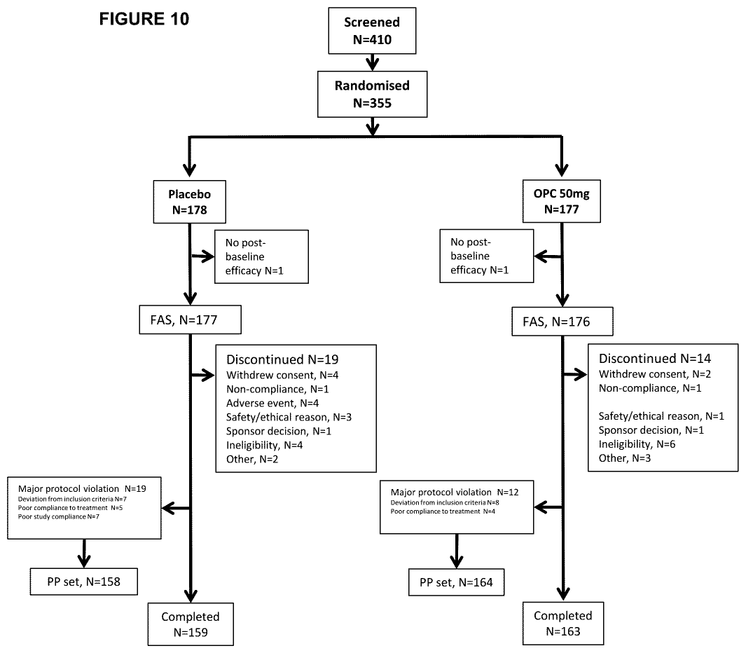

After a screening period of up to 4 weeks, eligible subjects were randomized to 1 of 2 treatment arms (opicapone (50 mg), or placebo) in a 1:1 ratio, and entered a 24-week placebo-controlled, parallel-group, double-blind period (FIGS. 9 and 10).

Study treatment was administered in combination with existing treatment of L-DOPA/DDCI (Table 1).

Randomization occurred at Visit 2 after confirmation of eligibility. Subjects were randomized 1:1 to either opicapone or placebo. Preferably, no stratification was performed during the randomization.

Subjects were centrally assigned to randomized study treatment using an Interactive Voice/Web Response System (IVRS/IWRS). Before the study was initiated, the telephone number and call-in directions for the IVRS and/or the log in information and directions for the IWRS was provided to each study center.

The study was a double-blind study with limited access to the randomization code. The investigational treatment and placebo capsules were identical in physical appearance. The treatment each subject receives was not disclosed to the investigator, study center staff, subject, sponsor, or study vendors. The treatment codes were held by the IVRS/IWRS vendor.

A Post-study Visit (PSV) was performed approximately 2 weeks after the End-of-Study Visit (EOS) or Early Discontinuation Visit (EDV).

At the end of the double-blind period, a number of subjects enter an additional 1-year, open-label period, at the discretion of the investigator, in which all subjects are treated with opicapone (50 mg). The double-blind period was unblinded after database lock for the purpose of data analyses; however, subjects and sites remain blinded to their double-blind treatment until the end of the open-label phase (ongoing).

### Dose Modifications

Levodopa/DDCI dose was adjusted if medically necessary, for example, due to motor complications, such as troublesome or dangerous dyskinesia. Modification of the dose of study treatments (opicapone or placebo) was not permitted.

### Statistical Methods

The primary efficacy analysis was performed after all subjects had completed the trial. Unblinding of the double-blind trial was performed after database lock for the purpose of data analyses.

D. Symptomatic Readouts

### Movement Disorder Society Unified Parkinson's Disease Rating Scales

The Movement Disorder Society Unified Parkinson's Disease Rating Scale (MDS-UPDRS) (Goetz C. et al., Mov. Disord., 2008, 23, 2129-70) is the MDS-sponsored revision and expansion of the widely used Unified Parkinson's Disease Rating Scale (UPDRS). The MDS-UPDRS was administered as follows:
MDS-UPDRS Part I (Non-Motor Aspects of Experiences of Daily Living).
MDS-UPDRS Part II (Motor Aspects of Experiences of Daily Living).
MDS-UPDRS Part III (Motor Examination) Scores (Primary Efficacy Endpoint).
MDS-UPDRS Part IV (Motor Complications).

Methods for calculation of the total score, as well as analysis of the sub-section are known to the skilled person.

### Hoehn & Yahr Staging

The modified Hoehn and Yahr scale is used to describe the progression of Parkinson disease symptoms. The original version (Hoehn M., Yahr M., Neurology, 1967, 17, 427-42) included stages 1 to 5.

### Schwab and England Scale

The Schwab and England activities of daily living scale is a measure of daily function on a scale of 0 (indicating worst possible function) to 100 (indicating no impairment) (Schwab R., England A., 1969;152-7).

### Parkinson's Disease Sleep Scale

The Parkinson's Disease Sleep Scale Version 2 (PDSS-2) is a specific scale for the assessment of sleep disturbances in subjects with Parkinson's disease (Chaudhuri K. et al., Mov. Disord., 2006, 21, 916-23). The PDSS-2 was used to investigate night-time symptoms and should be completed after the Clinical Global Impression.

### Non-Motor Symptoms Scale

Non-motor symptoms have a great impact on patients with Parkinson's disease. The MDS Non-motor Symptoms Scale (MDS-NMSS) is an instrument specifically designed for the comprehensive assessment of non-motor symptoms in patients with Parkinson's disease (Chaudhuri K. et al., Mov. Disord., 2007, 22, 1901-11). The MDS-NMSS contains 9 dimensions: cardiovascular, sleep/fatigue, mood/cognition, perceptual problems, attention/memory, gastrointestinal, urinary, sexual function, and miscellany in a 30-item scale.

The MDS-NMSS was completed after the Clinical Global Impression.

### Parkinson's Disease Questionnaire

Subjects assessed various aspects of functioning and well-being adversely affected by Parkinson's disease by completing the Parkinson's Disease Questionnaire (PDQ-39). The PDQ-39 is the most widely used Parkinson's disease-specific measure of health status. It contains 39 questions, covering 8 aspects of quality of life (mobility, activities of daily living [ADL], emotions, stigma, social support, cognitions, communication and bodily discomfort). The instrument was developed on the basis of interviews with people diagnosed with the disease and has been widely validated (Peto V et al., Qual. Life Res., 1995, 4, 241-8;Jenkinson Cet al., Age Ageing, 1997,26, 353-7).

The PDQ-39 scale was completed after the Clinical Global Impression.

9-item ‘Wearing’ Off Patient Card Questionnaire

The ‘Wearing’ Off Patient Card Questionnaire (WOQ-9) (Stacy M., et al., Clin. Neuropharmacol., 2006, 29, 312-21), lists 9 symptoms related to Parkinson's disease: tremor, mood changes, any slowness, reduced dexterity, any stiffness, anxiety/panic attacks, cloudy mind/slow thinking, muscle cramping and pain/aching. Patients are asked to mark which of these symptoms they are experiencing and whether they usually improve after the next dose of treatment. If a symptom is reported to improve after the following dose of medication, this is considered a “positive response.”

### Clinical Global Impression

The Clinical Global Impression (CGI) of improvement (CGI-I) is a 7-point scale that assesses how much the patient's illness has improved or worsened relative to baseline: very much improved, much improved, minimally improved, no change, minimally worse, much worse, or very much worse. Patients with ‘improvement’ are those rated as very much improved, much improved or minimally improved.

For an individual subject, the CGI scales was preferably scored by the same investigator/rater throughout the study.

### Patient's Global Impression

The Patient's Global Impression (PGI) improvement scale (PGI-I) consists of items from the CGI adapted to the patient. The Investigator rates the subject before the subject makes his/her own assessment. Preferably, the subject assess their own condition relative to their condition at admission to the study, using the PGI improvement scale (PGI-I).

E. Clinical Trial

### Opicapone Treatment

Opicapone was synthesised as described in WO 2013/089573 and formulated into 50 mg capsules as described in WO 2010/114405. Study treatment (opicapone or matching placebo) was taken orally once daily in the evening at least 1 hour after the last daily dose of L-DOPA/DDCI (considered the bedtime dose).

There was no change to the subject's L-DOPA/DDCI regimen throughout the double-blind period of the study unless adjustment was necessary for subject safety. In the open-label period (ongoing), L-DOPA/DDCI dose adjustments and new anti-Parkinson's disease drugs was permitted if necessary for subject safety and/or to treat a worsening of the patient's condition; adjustments for any other reason were discouraged.

TABLE 1
Study Treatment Details

| | | |
|---|---|---|
| Study Treatment Name: | Opicapone 50 mg | Placebo to match |
| Dosage Formulation: | Capsule | Capsule |
| Unit Dose Strength: | 50 mg | Not applicable |

| | |
|---|---|
| Route of Administration: | Oral |
| Dosing Instructions: | Swallow whole with water, once daily at |
| | bedtime at least 1 hour after L-DOPA/DDCI |
| Packaging and Labeling: | Study treatments is provided in suitable |
| | containers, which is labelled per country |
| | requirements. |
| Storage: | Keep out of the reach and sight of children |
| | and store at a temperature below 30° C. |
| | (inclusive) |
| Manufacturer: | BIAL - Portela & C^{a}, S.A., |
| | Coronado (S. Romão e S. Mamede), Portugal |

### Clinical Trial Design

The applicant performed a Phase III, multicenter, double-blind, placebo-controlled, parallel-group study evaluating the efficacy and safety of opicapone in patients with early idiopathic Parkinson's disease treated with levodopa/DDCI, with no signs of motor complications (e.g. fluctuations in the motor response and/or involuntary movements and/or dyskinesia). Patients in this study had early-stage Parkinson's disease with no motor complications; however, WOQ-9 and MDS-UPDRS Part IV was used to follow the emergence of any motor complications. Based on the WOQ-9 questionnaire, the proportion of subjects who experienced signs and symptoms of PD which improved post-study medication was high in the opicapone treated group for tremors (65.5%), slowness of movement (66.2%), stiffness (56.9%), dexterity (56.6%), and muscle cramping (28.3%). The proportion of subjects showing improvement post-study medication in the placebo group was similar to those observed for the placebo group except for dexterity which improved in 44.1% of placebo subjects. 355 subjects were randomized at an estimated 85 centers in 13 countries. 322 subjects completed the double-blind phase (FIG. 10). The study has an additional 52-week open-label extension (ongoing).

### Period 1—Screening (V1)

A screening visit took place within 4 weeks before Visit 2. Subject informed consent for the double-blind period was obtained using the informed consent form (ICF) before any study-related procedures were performed.

### Period 2—Double-Blind Period (V2 to V9)

At Visit 2, subjects maintained levodopa/DCCI therapy, which had been stable for at least 4 weeks. Eligible subjects were randomized to 1 of 2 treatment arms (opicapone (50 mg), or placebo) in a 1:1 ratio, and entered a 24-week double-blind trial. Study treatment was administered in combination with the subject's existing treatment of levodopa/DDCI.

The baseline characteristics of the subjects are shown in Table 2:

TABLE 2
Baseline Characteristics - patient characteristics

| | | Opicapone 50 mg | | Placebo | |
|---|---|---|---|---|---|
| | | (N = 177) | | (N = 178) | |
| Age (years) | Mean (SD) | 63.7 | (9.5) | 64.5 | (9.6) |
| Gender (Male) | n (%) | 109 | (61.6) | 121 | (68.0) |
| Caucasian | n (%) | 175 | (98.9) | 177 | (99.4) |
| Weight (Kg) | Mean (SD) | 79.6 | (13.1) | 80.4 | (14.4) |
| BMI (kg/m²) | Mean (SD) | 27.8 | (4.3) | 27.5 | (3.9) |
| PD duration (years) | Mean (SD) | 3.0 | (1.2) | 2.9 | (1.6) |
| Hoehn & Yahr | | | | | |
| Stage 1 | n (%) | 13 | (7.3) | 8 | (4.5) |
| Stage 1.5 | n (%) | 23 | (13.0) | 17 | (9.6) |
| Stage 2 | n (%) | 119 | (67.2) | 124 | (69.7) |
| Stage 2.5 | n (%) | 22 | (12.4) | 29 | (16.3) |
| MDS-UPDRS III | Mean (SD) | 32.7 | (10.9) | 34.4 | (11.7) |

The baseline characteristics of the subjects' treatment regimens are shown in Table 3:

TABLE 3
Baseline Characteristics - treatment regimens

| | | Opicapone 50 mg | | Placebo | |
|---|---|---|---|---|---|
| | | (N = 177) | | (N = 178) | |
| L-DOPA amount | mg (SD) | 386.8 | (137.2) | 391.4 | (111.4) |
| L-DOPA monotherapy | n (%) | 76 | (42.9) | 76 | (42.7) |
| L-DOPA plus | n (%) | 101 | (57.1) | 102 | (57.3) |
| plus MAO-Bi or DA | n (%) | 92 | (91.1) | 92 | (90.2) |
| plus MAO and DA | n (%) | 25 | (24.8) | 21 | (20.6) |
| plus DA only | n (%) | 55 | (54.5) | 51 | (50.0) |
| plus MAO only | n (%) | 12 | (11.9) | 20 | (19.6) |
| plus Amantadine | n (%) | 16 | (15.8) | 18 | (17.6) |
| DA | n (%) | 77 | (76.2) | 71 | (69.6) |
| Pramipexole | n (%) | 46 | (45.5) | 46 | (45.1) |
| Ropinirole | n (%) | 24 | (23.8) | 18 | (17.6) |
| Rotigotine | n (%) | 5 | (5.0) | 7 | (6.9) |
| Piribidil | n (%) | 3 | (3.0) | 0 | (0.0) |
| MAO-Bi | n (%) | 40 | (39.6) | 42 | (41.2) |
| Rasagiline | n (%) | 35 | (34.7) | 32 | (31.4) |
| Safinamide | n (%) | 3 | (3.0) | 6 | (5.9) |
| Selegiline | n (%) | 2 | (2.0) | 4 | (3.9) |

Subjects continued study treatment in combination with levodopa/DDCI and attended 7 study visits (V2 to V8) at 4-week intervals.

The End-of-Study (EOS) visit was Visit 9, for subjects who did not continue into the open-label period; otherwise subjects continue into the open-label period (ongoing). In the event of early discontinuation from the study, the subject attended an Early Discontinuation Visit (EDV).

A Post-study Visit (PSV) was performed at the study center approximately 2 weeks after the EQS visit or the EDV for subjects who did not enter the open-label period (ongoing).

### Primary Efficacy Analysis

The primary efficacy parameter, change from baseline (Visit 2) in MDS-UPDRS Part III total score at the end of the double-blind period (Visit 9), was analyzed using a Mixed Model Repeated Measures (MMRM) approach with fixed effects for baseline, center/country, (randomized) treatment, visit, treatment by visit interaction and baseline by visit interaction, and subject as a random effect. Difference between treatment groups (opicapone versus placebo) was estimated from the model.

At the end of the 24-week double-blind period, subjects treated with opicapone presented with a statistically significant lower motor disability compared to placebo, as shown in FIG. 1 and Table 4:

TABLE 4
Primary endpoint - change in MDS-UPDRS Part
III total score from baseline to 24 weeks

| | | OPC 50 mg | | Placebo | |
|---|---|---|---|---|---|
| | | (N = 176) | | (N = 177) | |
| DB Baseline | Mean (SD) | 32.7 | (10.9) | 34.4 | (11.7) |
| Week 24 | Mean (SD) | 27.2 | (12.4) | 30.0 | (14.3) |
| Change from Baseline | Mean (SD) | −5.8 | (8.4) | −3.7 | (9.8) |
| Estimates from | LS mean (SE) | −6.5 | (0.7) | −4.3 | (0.7) |
| MMRM | | | | | |

| | |
|---|---|
| LS Mean Diff. | −2.2 (0.9) |
| (SE) (95% CI) | (−3.9, −0.5) |
| P = value | 0.010 |

DB = Double-blind; MDS-UPDRS = Movement Disorder Society Unified Parkinson's Disease Rating Scale; N = Number of patients in the analysis set; n = number of patients with data; SD = Standard Deviation; OPC = Opicapone 50 mg; MMRM = Mixed Model for Repeated Measures; LS = Least Squares; SE = Standard Error; CI = Confidence Interval; Diff. = Difference; The significance level is 5% two-sided.

The longitudinal data confirm the magnitude of the effect, compared to placebo, increased over time, as shown in FIG. 2 and Table 5:

TABLE 5
Primary endpoint - longitudinal data with change in MDS-
UPDRS Part III total score from baseline to 24 weeks

| | | OPC 50 mg | | Placebo | |
|---|---|---|---|---|---|
| | | N = 176 | | N = 177 | |
| DB Baseline | Mean (SD) | 32.7 | (10.9) | 34.4 | (11.7) |
| Change to Week 2 | LS mean (SE) | −4.6 | (0.7) | −3.8 | (0.7) |
| Estimates from | LS Mean Diff. | −0.8 (0.8) | | | |
| MMRM | (SE) (95% CI); | (−2.4, 0.8); p = 0.336 | | | |
| | P value | | | | |
| Change to Week 4 | LS mean (SE) | −6.0 | (0.7) | −4.8 | (0.7) |
| Estimates from | LS Mean Diff. | −1.2 (0.8) | | | |
| MMRM | (SE) (95% CI); | (−2.8, 0.4); p = 0.147 | | | |
| | P value | | | | |
| Change to Week 12 | LS mean (SE) | −6.2 | (0.7) | −4.6 | (0.7) |
| Estimates from | LS Mean Diff. | −1.7 (0.9) | | | |
| MMRM | (SE) (95% CI); | (−3.3, 0.0); p = 0.051 | | | |
| | P value | | | | |
| Change to Week 24 | LS mean (SE) | −6.5 | (0.7) | −4.3 | (0.7) |

| | | |
|---|---|---|
| Estimates from | LS Mean Diff. | −2.2 (0.9) |
| MMRM | (SE) (95% CI); | (−3.9, −0.5); p = 0.010 |
| | P value | |

DB = Double-blind; MDS-UPDRS = Movement Disorder Society Unified Parkinson's Disease Rating Scale; N = Number of patients in the analysis set; n = number of patients with data; SD = Standard Deviation; OPC = Opicapone 50 mg; MMRM = Mixed Model for Repeated Measures; LS = Least Squares; SE = Standard Error; CI = Confidence Interval; Diff. = Difference; The significance level is 5% two-sided.

At every time point, the effect for opicapone was greater in magnitude than the placebo. The fact the magnitude of the effect increased over time suggests that less significant effects might become significant over extended treatments as the therapeutic effect is maintained (or increased) and the placebo effect subsides. Likewise, the placebo effect at early time points might disguise the magnitude of the therapeutic effect.

### Sensitivity Analysis

A sensitivity analysis was performed on the primary endpoint using an analysis of covariance (ANCOVA) approach, with fixed effects for baseline, center/country and (randomized) treatment, or using MMRM analysis. Missing data was imputed using a multiple imputation method for the sensitivity analysis of the primary endpoint only.

### 5 Secondary Efficacy Analysis

A similar MMRM analysis used for the primary endpoint was used for relevant secondary efficacy endpoints in the double-blind period. The secondary endpoints included:
Change from baseline (Visit 2) to post-baseline visits during the double-blind trial in:
   MDS-UPDRS scores: Parts I, II, III and IV, and Part II+III total
   Modified Hoehn & Yahr staging total score during maximum ‘ON’ response
   Schwab and England scale score
   Parkinson's Disease Sleep Scale 2 (PDSS-2) total score
   MDS-Non-motor Symptom Scale (MDS-NMSS) total and subdomain scores
   Parkinson's Disease Questionnaire (PDQ-39) total and subdomain scores
   9-item Wearing-off questionnaire (WOQ9) total and sub-section (motor and non-motor) scores
Clinical Global Impression of Improvement (CGI-I)
Patient's Global Impression of Improvement (PGI-I)

At the end of the 24-week double-blind period, subjects treated with opicapone presented with a statistically significant lower MDS-UPDRS Part II+III total score compared to placebo, as shown FIG. 3a and Table 6:

TABLE 6
Change in MDS-UPDRS Part II +
III total score from baseline to week 24

| | | OPC 50 mg | | Placebo | |
|---|---|---|---|---|---|
| | | (N = 176) | | (N = 177) | |
| DB Baseline | Mean (SD) | 41.8 | (15.1) | 43.5 | (16.1) |
| Week 24 | Mean (SD) | 35.7 | (16.5) | 39.0 | (18.0) |
| Change from | Mean (SD) | −6.2 | (10.8) | −3.3 | (11.7) |
| Baseline | | | | | |
| Estimates from | LS mean (SE) | −7.4 | (1.02) | −4.6 | (1.01) |
| MMRM | | | | | |

| | |
|---|---|
| LS Mean Diff. (SE) | −2.8 (1.3) |
| (95% CI) | (−5.4, −0.2) |
| P = value | 0.036 |

DB = Double-blind; MDS-UPDRS = Movement Disorder Society Unified Parkinson's Disease Rating Scale; N = Number of patients in the analysis set; n = number of patients with data; SD = Standard Deviation; OPC = Opicapone 50 mg; MMRM = Mixed Model for Repeated Measures; LS = Least Squares; SE = Standard Error; CI = Confidence Interval; Diff. = Difference; The significance level is 5% two-sided.

The longitudinal data confirm the magnitude of the effect, compared to placebo, increased over time, as shown in FIG. 3b and Table 7:

TABLE 7
longitudinal data with change in MDS-UPDRS Part
II + III total score from baseline to 24 weeks

| | | OPC 50 mg | | Placebo | |
|---|---|---|---|---|---|
| | | (N = 176) | | (N = 177) | |
| DB Baseline | Mean (SD) | 41.8 | (15.1) | 43.5 | (16.1) |
| Change to Week 4 | LS mean (SE) | −7.3 | (0.8) | −5.9 | (0.8) |
| Estimates from | LS Mean Diff. | −1.4 (0.9) | | | |
| MMRM | (SE) (95% CI); | (−3.3, 0.4); p = 0.131 | | | |
| | P value | | | | |
| Change to Week 12 | LS mean (SE) | −7.4 | (0.9) | −5.7 | (0.8) |
| Estimates from | LS Mean Diff. | −1.7 (1.06) | | | |
| MMRM | (SE) (95% CI); | (−3.8, 0.4); p = 0.109 | | | |
| | P value | | | | |
| Change to Week 24 | LS mean (SE) | −7.4 | (1.02) | −4.6 | (1.01) |

| | | |
|---|---|---|
| Estimates from | LS Mean Diff. | −2.8 (1.3) |
| MMRM | (SE) (95% CI); | (−5.4, −0.2); p = 0.036 |
| | P value | |

The magnitude of the effect for opicapone remained constant over time with the effect compared to placebo becoming significant as the placebo effect subsided.

Furthermore, the results shown in FIG. 4a demonstrate a significantly higher proportion of opicapone-treated patients reported an improvement in their clinical condition (PGI-I score) with 57.9% showing an improvement compared to 45.6% of placebo-treated patients (p=0.026). A similar trend is shown in FIG. 4b when the clinical condition was assessed by a clinician (CGI-I score) with 50.3% showing an improvement compared to 46.2% of placebo-treated patients (p=0.493).

At the end of the 24-week double-blind period, subjects treated with opicapone presented with a statistically significant improvement compared to placebo-treated patients in the PDSS-2 (p=0.039) with no worsening observed in the opicapone-treated group, as shown FIG. 5 and Table 8:

TABLE 8
Change in PDSS-2 total score from baseline to week 24

| | | OPC 50 mg | | Placebo | |
|---|---|---|---|---|---|
| | | (N = 176) | | (N = 177) | |
| DB Baseline | Mean (SD) | 12.4 | (8.7) | 11.9 | (8.6) |
| Week 24 | Mean (SD) | 11.6 | (7.0) | 12.7 | (9.2) |
| Change from | Mean (SD) | −0.4 | (7.4) | 1.1 | (8.3) |
| Baseline | | | | | |
| Estimates from | LS mean (SE) | 0.0 | (0.6) | 1.4 | (0.57) |

MMRM

| | |
|---|---|
| LS Mean Diff. (SE) | −1.5 (0.7) |
| (95% CI) | (−1.9, −0.1) |
| P = value | 0.039 |

DB = Double-blind; PDSS = Parkinson's Disease Sleep Scale; N = Number of patients in the analysis set; n = number of patients with data; SD = Standard Deviation; OPC = Opicapone 50 mg; MMRM = Mixed Model for Repeated Measures; LS = Least Squares; SE = Standard Error; CI = Confidence Interval; Diff. = Difference; The significance level is 5% two-sided.

At the end of the 24-week double-blind period, subjects treated with opicapone presented with a positive trend towards a therapeutic effect for the MDS-UPDRS Part 11 total score, as shown FIG. 6a and Table 9:

TABLE 9
Change in MDS-UPDRS Part II total
score from baseline to week 24

| | | OPC 50 mg | | Placebo | |
|---|---|---|---|---|---|
| | | (N = 176) | | (N = 177) | |
| DB Baseline | Mean (SD) | 9.1 | (5.8) | 9.1 | (6.1) |
| Week 24 | Mean (SD) | 8.5 | (4.6) | 9.0 | (5.8) |
| Change from | Mean (SD) | −0.4 | (4.1) | 0.4 | (3.7) |
| Baseline | | | | | |
| Estimates from | LS mean (SE) | −0.4 | (0.34) | 0.3 | (0.33) |

MMRM

| | |
|---|---|
| LS Mean Diff. (SE) | −0.7 (0.4) |
| (95% CI) | (−1.5, 0.2) |
| P = value | 0.120 |

DB = Double-blind; MDS-UPDRS = Movement Disorder Society Unified Parkinson's Disease Rating Scale; N = Number of patients in the analysis set; n = number of patients with data; SD = Standard Deviation; OPC = Opicapone 50 mg; MMRM = Mixed Model for Repeated Measures; LS = Least Squares; SE = Standard Error; CI = Confidence Interval; Diff. = Difference; The significance level is 5% two-sided.

The longitudinal data confirm the magnitude of the effect, compared to placebo, increased over time, as shown in FIG. 6b and Table 10:

TABLE 10
longitudinal data with change in MDS-UPDRS Part
II total score from baseline to 24 weeks

| | | OPC 50 mg | | Placebo | |
|---|---|---|---|---|---|
| | | (N = 176) | | (N = 177) | |
| DB Baseline | Mean (SD) | 9.1 | (5.8) | 9.1 | (6.1) |
| Change to Week 4 | LS mean (SE) | −0.8 | (0.25) | −0.6 | (0.25) |
| Estimates from | LS Mean Diff. | −0.2 (0.3) | | | |
| MMRM | (SE) (95% CI); | (−0.8, 0.4); p = 0.500 | | | |
| | P value | | | | |
| Change to Week 12 | LS mean (SE) | −0.7 | (0.29) | −0.6 | (0.29) |
| Estimates from | LS Mean Diff. | −0.2 (0.4) | | | |
| MMRM | (SE) (95% CI); | (−0.9, 0.6); p = 0.667 | | | |
| | P value | | | | |
| Change to Week 24 | LS mean (SE) | −0.4 | (0.34) | 0.3 | (0.33) |

| | | |
|---|---|---|
| Estimates from | LS Mean Diff. | −0.7 (0.4) |
| MMRM | (SE) (95% CI); | (−1.5, 0.2); p = 0.120 |
| | P value | |

DB = Double-blind; MDS-UPDRS = Movement Disorder Society Unified Parkinson's Disease Rating Scale; N = Number of patients in the analysis set; n = number of patients with data; SD = Standard Deviation; OPC = Opicapone 50 mg; MMRM = Mixed Model for Repeated Measures; LS = Least Squares; SE = Standard Error; CI = Confidence Interval; Diff. = Difference; The significance level is 5% two-sided.

This suggests larger groups or longer treatments might achieve statistical significance.

At the end of the 24-week double-blind period, subjects treated with opicapone presented with a positive trend towards a therapeutic effect for the NMSS, as shown FIG. 7 and Table 11:

TABLE 11
Change in the NMSS from baseline to week 24

| | | OPC 50 mg | | Placebo | |
|---|---|---|---|---|---|
| | | (N = 176) | | (N = 177) | |
| DB Baseline | Mean (SD) | 19.9 | (17.2) | 19.2 | (17.3) |
| Week 24 | Mean (SD) | 15.9 | (15.2) | 17.7 | (16.8) |
| Change from | Mean (SD) | −3.0 | (13.2) | −1.0 | (13.8) |
| Baseline | | | | | |
| Estimates from | LS mean (SE) | −1.4 | (0.96) | 0.5 | (0.96) |

MMRM

| | |
|---|---|
| LS Mean Diff. (SE) | −−2.0 (1.2) |
| (95% CI) | (−4.4, 0.4) |
| P = value | 0.102 |

DB = Double-blind; NMSS = Non-motor Symptoms Scale; N = Number of patients in the analysis set; n = number of patients with data; SD = Standard Deviation; OPC = Opicapone 50 mg; MMRM = Mixed Model for Repeated Measures; LS = Least Squares; SE = Standard Error; CI = Confidence Interval; Diff. = Difference; The significance level is 5% two-sided.

At the end of the 24-week double-blind period, no significant effect was observed for the MDS-UPDRS Part I total score, as shown Table 12:

TABLE 12
Change in the MDS-UPDRS Part I total
score from baseline to week 24

| | | OPC 50 mg | | Placebo | |
|---|---|---|---|---|---|
| | | (N = 176) | | (N = 177) | |
| DB Baseline | Mean (SD) | 6.6 | (4.6) | 6.8 | (5.0) |
| Week 24 | Mean (SD) | 6.7 | (4.6) | 6.5 | (5.0) |
| Change from | Mean (SD) | 0.3 | (3.9) | 0.0 | (3.4) |
| Baseline | | | | | |
| Estimates from | LS mean (SE) | 0.4 | (0.28) | 0.2 | (0.28) |

MMRM

| | |
|---|---|
| LS Mean Diff. (SE) | −0.2 (0.4) |
| (95% CI) | (−0.5, 0.9) |
| P = value | 0.512 |

DB = Double-blind; MDS-UPDRS = Movement Disorder Society Unified Parkinson's Disease Rating Scale; N = Number of patients in the analysis set; n = number of patients with data; SD = Standard Deviation; OPC = Opicapone 50 mg; MMRM = Mixed Model for Repeated Measures; LS = Least Squares; SE = Standard Error; CI = Confidence Interval; Diff. = Difference; The significance level is 5% two-sided.

At the end of the 24-week double-blind period, no significant effect was observed for the MDS-UPDRS Part IV total score, as shown Table 13:

TABLE 13
Change in the MDS-UPDRS Part IV total
score from baseline to week 24

| | | OPC 50 mg | Placebo |
|---|---|---|---|
| | | (N = 176) | (N = 177) |
| DB Baseline | Mean (SD) | 0.0 (0.0) | 0.0 (0.4) |
| Week 24 | Mean (SD) | 0.2 (0.8) | 0.3 (1.1) |
| Change from | Mean (SD) | 0.2 (0.8) | 0.3 (1.1) |
| Baseline | | | |
| Estimates from | LS mean (SE) | 0.3 (0.1) | 0.4 (0.1) |
| MMRM | | | |

| | |
|---|---|
| LS Mean Diff. (SE) | −0.11 (0.1) |
| (95% CI) | (−0.3, 0.1) |
| P = value | 0.220 |

DB = Double-blind; MDS-UPDRS = Movement Disorder Society Unified Parkinson's Disease Rating Scale; N = Number of patients in the analysis set; n = number of patients with data; SD = Standard Deviation; OPC = Opicapone 50 mg; MMRM = Mixed Model for Repeated Measures; LS = Least Squares; SE = Standard Error; CI = Confidence Interval; Diff. = Difference; The significance level is 5% two-sided.

Although the patient population selected for treatment according to the present invention does not suffer from motor complications, the trial assessed the emergence of motor complications throughout the double-blind period. At the end of the 24-week double-blind period, a lower proportion of opicapone-treated patients reported motor complications (5.5%) compared to placebo-treated patients (9.8%), as shown in FIG. 8.

At the end of the 24-week double-blind period, no significant effect was observed for the PDQ-39 total score, as shown Table 14:

TABLE 14
Change in the PDQ-39 total score from baseline to week 24

| | | OPC 50 mg | | Placebo | |
|---|---|---|---|---|---|
| | | (N = 176) | | (N = 177) | |
| DB Baseline | Mean (SD) | 16.4 | (12.9) | 15.6 | (13.7) |
| Week 24 | Mean (SD) | 15.4 | (12.5) | 14.5 | (13.1) |
| Change from | Mean (SD) | −0.29 | (7.4) | 0.31 | (8.1) |
| Baseline | | | | | |
| Estimates from | LS mean (SE) | 0.42 | (0.62) | 0.69 | (0.62) |

MMRM

| | |
|---|---|
| LS Mean Diff. (SE) | −0.26 (0.77) |
| (95% CI) | (−1.78, 1.25) |
| P = value | 0.733 |

DB = Double-blind; PDQ = Parkinson's Disease Questionnaire; N = Number of patients in the analysis set; n = number of patients with data; SD = Standard Deviation; OPC = Opicapone 50 mg; MMRM = Mixed Model for Repeated Measures; LS = Least Squares; SE = Standard Error; CI = Confidence Interval; Diff. = Difference; The significance level is 5%
two-sided.

The magnitude of the changes in both groups (opicapone and placebo) for the MDS-UPDRS Part I, MDS-UPDRS Part IV or PDQ-39 total scores were all less than 0.4 points. Therefore, longer treatment periods or larger trials may be required to observe significant effects in these symptoms.

The proportion of subjects with an improvement from baseline in CGI-I (preferably relative to before the beginning of treatment) and PGI-I (preferably relative to their admission to the study) scores at the end of the double blind period (Visit 9) endpoint was analysed using logistic regression, with (randomized) treatment included in the model.

The results shown in FIG. 4a demonstrate a significantly higher proportion of opicapone-treated patients reported an improvement in their clinical condition (PGI-I score) with 57.9% showing an improvement compared to 45.6% of placebo-treated patients (p=0.026). A similar trend is shown in FIG. 4b when the clinical condition was assessed by a clinician (the CGI-I score) with 50.3% showing an improvement compared to 46.2% of placebo-treated patients (p 0.493).

During the double blind phase, opicapone shows good tolerability and a low incidence of Adverse Events (AEs) including Treatment Emergent Adverse Events, as shown Table 15 and Table 16:

TABLE 15
Treatment Emergent Adverse Events
(TEAEs) in the double blind phase

| | Opicapone 50 mg | | Placebo | |
|---|---|---|---|---|
| | (N = 177) | | (N = 178) | |
| Any TEAE; n (%) | 84 | (47.5) | 84 | (47.2) |
| Any TEAE of SI; n (%) | 13 | (7.3) | 12 | (6.7) |
| Any TEAE by Maximum Severity | | | | |
| Mild | 53 | (29.9) | 49 | (27.5) |
| Moderate; n (%) | 25 | (14.1) | 31 | (17.4) |
| Severe; n (%) | 6 | (3.4) | 4 | (2.2) |
| Any TEAE by Worst Relationship | | | | |
| Related; n (%) | 18 | (10.2) | 24 | (13.5) |
| Definitely Related; n (%) | 2 | (1.1) | 5 | (2.8) |
| Probably Related; n (%) | 6 | (3.4) | 4 | (2.2) |
| Possibly Related; n (%) | 10 | (5.6) | 15 | (8.4) |
| Not Related; n (%) | 66 | (37.3) | 60 | (33.7) |
| Unlikely Related; n (%) | 2 | (1.1) | 6 | (3.4) |
| Not Related; n (%) | 64 | (36.2) | 54 | (30.3) |
| Any Serious TEAE; n (%) | 9 | (5.1) | 5 | (2.8) |
| Any TEAE leading to Death; n (%) | 1 | (0.6) | 3 | (1.7) |
| Any TEAE leading to IMP | 1 | (0.6) | 4 | (2.2) |
| interruption; n (%) | | | | |
| Any TEAE leading to Withdrawal; n | 2 | (1.1) | 7 | (3.9) |
| (%) | | | | |

TABLE 16
Specific TEAEs in the double blind phase

| | Opicapone 50 mg | | Placebo | |
|---|---|---|---|---|
| | (N = 177) | | (N = 178) | |
| Any TEAE; n (%) | 84 | (47.5) | 84 | (47.2) |
| Nervous System Disorders; n (%) | 24 | (13.6) | 29 | (16.3) |
| On and off phenomenon; n (%) | 8 | (4.5) | 5 | (2.8) |
| Tremor; n (%) | 2 | (1.1) | 7 | (3.9) |
| Dyskinesia; n (%) | 3 | (1.7) | 4 | (2.2) |
| Dizziness; n (%) | 2 | (1.1) | 4 | (2.2) |
| Headache; n (%) | 1 | (0.6) | 5 | (2.8) |
| Infections and Infestations; n (%) | 28 | (15.8) | 23 | (12.9) |
| COVID-19; n (%) | 15 | (8.5) | 8 | (4.5) |
| Upper respiratory tract infection; | 1 | (0.6) | 4 | (2.2) |
| n (%) | | | | |
| Urinary tract infection; n (%) | 4 | (2.3) | 0 | (0.0) |
| Musculoskeletal and connective tissue | 18 | (10.2) | 14 | (7.9) |
| disorders; n (%) | | | | |
| Back pain; n (%) | 8 | (4.5) | 2 | (1.1) |
| Arthralgia; n (%) | 4 | (2.3) | 5 | (2.8) |
| Gastrointestinal disorders; n (%) | 12 | (6.8) | 18 | (10.1) |
| Nausea; n (%) | 4 | (2.3) | 3 | (1.7) |
| Vomiting; n (%) | 3 | (1.7) | 3 | (1.7) |
| General disorders; n (%) | 14 | (7.9) | 13 | (7.3) |
| Fatigue; n (%) | 5 | (2.8) | 4 | (2.2) |
| Oedema peripheral; n (%) | 4 | (2.3) | 2 | (1.1) |
| Psychiatric disorders; n (%) | 14 | (7.9) | 12 | (6.7) |
| Insomnia; n (%) | 4 | (2.3) | 1 | (0.6) |
| Vascular disorders; n (%) | 10 | (5.6) | 6 | (3.4) |
| Hypertension; n (%) | 5 | (2.8) | 2 | (1.1) |
| Injury, poisoning and procedural | 6 | (3.4) | 8 | (4.5) |
| complications; n (%) | | | | |
| Fall; n (%) | 4 | (2.3) | 3 | (1.7) |

Opicapone was found to be well tolerated in patients without clinically diagnosed motor complications with a more favourable safety profile than previous studies in patients with clinically diagnosed motor complications. Opicapone-treated patients saw no increase in treatment emergent adverse events including nervous system disorders such as dyskinesia. This is in stark contrast to the STRIDE-PD study where entacapone was associated with a shorter time to onset and increased frequency of dyskinesia compared to placebo. Therefore, the use of opicapone in treating Parkinson's disease patients without clinically diagnosed motor complications results in a surprising improvement in treatment efficacy without an increase in dyskinesia.

### Period 3—Open-Label Period (V9 to V15)

At the end of the double-blind period, subjects may enter an additional 1-year open-label period, in which all subjects are treated with opicapone (50 mg) in combination with their existing levodopa/DDCI. In the open-label period (ongoing), levodopa/DDCI dose adjustments and new anti-Parkinson's disease drugs is permitted if necessary for subject safety and/or to treat a worsening of the patient's condition; adjustments for any other reason are discouraged.

The doses of levodopa/DDCI therapy is recorded in the electronic case report form (eCRF).

The primary endpoint in the open-label phase is the change from open-label baseline (Visit 9) to the end of the open-label period (Visit 15) in MDS-UPDRS Part IV total score. The secondary endpoints include:
Change from double-blind baseline (Visit 2) and open-label baseline (Visit 9) to post-baseline visits in:
   MDS-UPDRS scores: Parts I, II, III and IV, and Part II+III total
   Modified Hoehn & Yahr staging total score during maximum ‘ON’ response
   Schwab and England scale score
   PDSS-2 total score
   MDS-NMSS total and subdomain scores
   PDQ-39 total and subdomain scores
   WOQ-9 total and sub-section (motor and non-motor) scores
CGI-I, preferably relative to their condition before the beginning of treatment
PGI-I, preferably relative to their condition at admission to the study

As explained above, the active treatment including opicapone (50 mg) showed good efficacy in the primary and several secondary end-points.

### Period 4—Double-Blind and Open-Label Periods (V2 to 15)

During the course of the double-blind period and open-label period (ongoing), the safety and tolerability of once-daily opicapone (50 mg) as an adjuvant to stable levodopa/DDCI therapy in patients with early stage Parkinson's disease is evaluated. The factors evaluated include:
Treatment-emergent adverse events (TEAEs) including serious adverse events (SAEs)
Laboratory safety tests (biochemistry, hematology, coagulation and urinalysis)
Physical and neurological examinations
Vital signs
12-lead ECG readings
Columbia-Suicide Severity Rating Scale (C-SSRS)
Modified Minnesota Impulsive Disorders Interview (mMIDI)

### REFERENCES

1. LeWitt P A. Levodopa therapy for Parkinson's disease: Pharmacokinetics and pharmacodynamics. Mov Disord. 2015; 30(1):64-72.
2. Olanow C W. Levodopa is the best symptomatic therapy for PD: Nothing more, nothing less. Mov Disord. 2019; 34(6):812-5.
3. Aquino C C, Fox S H. Clinical spectrum of levodopa-induced complications. Mov Disord. 2015; 30(1):80-9.
4. Chaudhuri K R, Jenner P, Antonini A. Should there be less emphasis on levodopa-induced dyskinesia in Parkinson's disease? Mov Disord. 2019; 34(6):816-9.
5. Stacy M. The wearing-off phenomenon and the use of questionnaires to facilitate its recognition in Parkinson's disease. J Neural Transm. 2010; 117(7):837-46.
6. Bjornestad A, Forsaa E B, Pedersen K F, Tysnes O B, Larsen J P, Alves G. Risk and course of motor complications in a population-based incident Parkinson's disease cohort. Parkinsonism Relat Disord. 2016; 22:48-53.
7. Scott N W, Macleod A D, Counsell C E. Motor complications in an incident Parkinson's disease cohort. Eur J Neurol. 2016; 23(2):304-12.
8. Kim H-J, Mason S, Foltynie T, Winder-Rhodes S, Barker R A, Williams-Gray C H. Motor Complications in Parkinson's Disease: 13-Year Follow-up of the CamPaIGN Cohort. Movement Disorders. 2020; 35(1):185-90.
9. LeWitt P A, Chaudhuri K R. Unmet needs in Parkinson disease: Motor and non-motor. Parkinsonism Relat Disord. 2020;80 Suppl 1:S7-S12.
10. Chapuis S, Ouchchane L, Metz O, Gerbaud L, Durif F. Impact of the motor complications of Parkinson's disease on the quality of life. Mov Disord. 2005; 20(2):224-30.
11. Hechtner M C, Vogt T, Zollner Y, Schroder S, Sauer J B, Binder H, et al. Quality of life in Parkinson's disease patients with motor fluctuations and dyskinesias in five European countries. Parkinsonism Relat Disord. 2014; 20:969-74.
12. Wu J, Lim E-C, Nadkarni N V, Tan E-K, Kumar P M. The impact of levodopa therapy-induced complications on quality of life in Parkinson's disease patients in Singapore. Scientific Reports. 2019;9(1):9248.
13. Soh S-E, Morris M E, McGinley J L. Determinants of health-related quality of life in Parkinson's disease: A systematic review. Parkinsonism & Related Disorders. 2011; 17(1):1-9.
14. Schrag A, Quinn N. Dyskinesias and motor fluctuations in Parkinson's disease. A community-based study. Brain. 2000;123(Pt 11):2297-305.
15. Foundation M J F. Capturing and Elevating the Patient Voice. Available at https://www.michaeljfox.org/foundation/news-detail.php?capturing-and-elevating-the-patient-voice 2014.
16. Pahwa R, Lyons K E. Levodopa-related wearing-off in Parkinson's disease: identification and management. Curr Med Res Opin. 2009; 25(4):841-9.
17. Hauser R A, McDermott M P, Messing S. Factors associated with the development of motor fluctuations and dyskinesias in Parkinson disease. Arch Neurol. 2006; 63(12):1756-60.
18. Olanow C W, Kieburtz K, Rascol O, Poewe W, Schapira A H, Emre M, et al. Factors predictive of the development of Levodopa-induced dyskinesia and wearing-off in Parkinson's disease. Mov Disord. 2013; 28(8):1064-71.
19. Kadastik-Eerme L, Taba N, Asser T, Taba P. Factors associated with motor complications in Parkinson's disease. Brain and behavior. 2017;7(10):e00837-e.
20. Stocchi F. The levodopa wearing-off phenomenon in Parkinson's disease: pharmacokinetic considerations. Expert Opin Pharmacother. 2006; 7(10):1399-407.
21. Stocchi F, Jenner P, Obeso J A. When Do Levodopa Motor Fluctuations First Appear in Parkinson's Disease? Eur Neurol. 2010; 63(5):257-66.
22. Olanow C W, Kieburtz K, Odin P, Espay A J, Standaert D G, Fernandez H H, et al. Continuous intrajejunal infusion of levodopa-carbidopa intestinal gel for patients with advanced Parkinson's disease: a randomised, controlled, double-blind, double-dummy study. Lancet Neurol. 2014; 13(2):141-9.
23. Antonini A, Stoessl A J, Kleinman L S, Skalicky A M, Marshall T S, Sail K R, et al. Developing consensus among movement disorder specialists on clinical indicators for identification and management of advanced Parkinson's disease: a multi-country Delphi-panel approach. Curr Med Res Opin. 2018; 34(12):2063-73.
24. Stocchi F, Giorgi L, Hunter B, Schapira A H. PREPARED: Comparison of prolonged and immediate release ropinirole in advanced Parkinson's disease. Mov Disord. 2011.
25. Schapira A H, Barone P, Hauser R A, Mizuno Y, Rascol O, Busse M, et al. Extended-release pramipexole in advanced Parkinson disease: a randomized controlled trial. Neurology. 2011; 77(8):767-74.
26. Katzenschlager R, Poewe W, Rascol O, Trenkwalder C, Deuschl G, Chaudhuri K R, et al. Apomorphine subcutaneous infusion in patients with Parkinson's disease with persistent motor fluctuations (TOLEDO): a multicentre, double-blind, randomised, placebo-controlled trial. Lancet Neurol. 2018; 17(9):749-59.
27. LeWitt P A, Lyons K E, Pahwa R. Advanced Parkinson disease treated with rotigotine transdermal system: PREFER Study. Neurology. 2007; 68(16):1262-7.
28. Antonini A, Tolosa E, Mizuno Y, Yamamoto M, Poewe W H. A reassessment of risks and benefits of dopamine agonists in Parkinson's disease. Lancet Neurol. 2009; 8(10):929-37.
29. Jenner P, Mori A, Aradi S D, Hauser R A. Istradefylline—a first generation adenosine A2A antagonist for the treatment of Parkinson's disease. Expert Review of Neurotherapeutics. 2021:null-null.
30. Elmer L W, Juncos J L, Singer C, Truong D D, Criswell S R, Parashos S, et al. Pooled Analyses of Phase III Studies of ADS-5102 (Amantadine) Extended-Release Capsules for Dyskinesia in Parkinson's Disease. CNS Drugs. 2018; 32(4):387-98.
31. Fox S H, Katzenschlager R, Lim S Y, Barton B, de Bie R M A, Seppi K, et al. International Parkinson and movement disorder society evidence-based medicine review: Update on treatments for the motor symptoms of Parkinson's disease. Mov Disord. 2018; 33(8):1248-66.
32. Keating G M, Lyseng-Williamson K A. Tolcapone: a review of its use in the management of Parkinson's disease. CNS Drugs. 2005; 19(2):165-84.
33. Kaakkola S. Problems with the present inhibitors and a relevance of new and improved COMT inhibitors in Parkinson's disease. Int Rev Neurobiol. 2010; 95:207-25.
34. Kiss L E, Soares-da-Silva P. Medicinal chemistry of catechol O-methyltransferase (COMT) inhibitors and their therapeutic utility. J Med Chem. 2014; 57(21):8692-717.
35. Cotzias G C. L-Dopa for Parkinsonism. N Engl J Med. 1968;278(11):630.
36. Fahn S, Oakes D, Shoulson I, Kieburtz K, Rudolph A, Lang A, et al. Levodopa and the progression of Parkinson's disease. N Engl J Med. 2004; 351(24):2498-508.
37. Verschuur C V M, Suwijn S R, Boel J A, Post B, Bloem B R, van Hilten J J, et al. Randomized Delayed-Start Trial of Levodopa in Parkinson's Disease. New England Journal of Medicine. 2019; 380(4):315-24.
38. Hauser R A. Levodopa: past, present, and future. Eur Neurol. 2009; 62(1):1-8.
39. Calne D B, Reid J L, Vakil S D, Rao S, Petrie A, Pallis C A, et al. Idiopathic Parkinsonism treated with an extracerebral decarboxylase inhibitor in combination with levodopa. Br Med J. 1971; 3(5777):729-32.
40. Nutt J G, Woodward W R, Anderson J L. The effect of carbidopa on the pharmacokinetics of intravenously administered levodopa: the mechanism of action in the treatment of parkinsonism. Ann Neurol. 1985; 18(5):537-43.
41. Lieberman A, Goodgold A, Jonas S, Leibowitz M. Comparison of dopa decarboxylase inhibitor (carbidopa) combined with levodopa and levodopa alone in Parkinson's disease. Neurology. 1975; 25(10):911-6.
42. Markham C, Diamond S G, Treciokas U. Carbidopa in Parkinson disease and in nausea and vomiting of levodopa. Arch Neurol. 1974; 31(2):128-33.
43. Gershanik O S. Improving 1-dopa therapy: The development of enzyme inhibitors. Movement Disorders. 2015; 30(1):103-13.
44. Deleu D, Northway M G, Hanssens Y. Clinical pharmacokinetic and pharmacodynamic properties of drugs used in the treatment of Parkinson's disease. Clin Pharmacokinet. 2002; 41(4):261-309.
45. Karhunen T, Tilgmann C, Ulmanen I, Julkunen I, Panula P. Distribution of catechol-O-methyltransferase enzyme in rat tissues. J Histochem Cytochem. 1994; 42(8):1079-90.
46. Nutt J G, Woodward W R, Gancher S T, Merrick D. 3-O-methyldopa and the response to levodopa in Parkinson's disease. Annals of Neurology. 1987; 21:584-8.
47. Kaakkola S. Clinical pharmacology, therapeutic use and potential of COMT inhibitors in Parkinson's disease. Drugs. 2000; 59(6):1233-50.
48. Bonificio M J, Palma P N, Almeida L, Soares-da-Silva P. Catechol-O-methyltransferase and its inhibitors in Parkinson's disease. CNS Drug Rev. 2007; 13(3):352-79.
49. Kaakkola S, Gordin A, Mannisto P T. General properties and clinical possibilities of new selective inhibitors of catechol O-methyltransferase. Gen Pharmacol. 1994; 25(5):813-24.
50. Artusi C A, Sarro L, Imbalzano G, Fabbri M, Lopiano L. Safety and efficacy of tolcapone in Parkinson's disease: systematic review. Eur J Clin Pharmacol. 2021.
51. Olanow C W, Watkins P B. Tolcapone: an efficacy and safety review (2007). Clin Neuropharmacol. 2007; 30(5):287-94.
52. Kiss L E, Ferreira H S, Torrao L, Bonifacio M J, Palma P N, Soares-da-Silva P, et al. Discovery of a long-acting, peripherally selective inhibitor of catechol-O-methyltransferase. J Med Chem. 2010; 53(8):3396-411.
53. Palma P N, Bonifacio M J, Loureiro A I, Soares-da-Silva P. Computation of the binding affinities of catechol-O-methyltransferase inhibitors: multisubstate relative free energy calculations. J Comput Chem. 2012; 33(9):970-86.
54. Bonifacio M C, Torrao M, Loureiro A I, Wright L C, Soares-Da-Silva P. Opicapone: characterization of a novel peripheral long-acting catechol-O-methyltransferase inhibitor. Parkinsonism Relat Disord 2012;18(suppl 2).
55. Bonifacio M J, Torrao L, Loureiro A I, Palma P N, Wright L C, Soares-da-Silva P. Pharmacological profile of opicapone, a third-generation nitrocatechol catechol-O-methyl transferase inhibitor, in the rat. Br J Pharmacol. 2015; 172(7):1739-52.
56. Bonifacio M J, Sutcliffe J S, Torrao L, Wright L C, Soares-da-Silva P. Brain and peripheral pharmacokinetics of levodopa in the cynomolgus monkey following administration of opicapone, a third generation nitrocatechol COMT inhibitor. Neuropharmacology. 2014; 77:334-41.
57. Bonifacio M J, Sousa F, Soares-da-Silva P. Opicapone enhances the reversal of MPTP-induced Parkinson-like syndrome by levodopa in cynomolgus monkeys. Eur J Pharmacol. 2021; 892:173742.
58. Tissot R, Bartholini G, Pletscher A. Drug-induced changes of extracerebral dopa metabolism in man. Arch Neurol. 1969; 20(2):187-90.
59. Rocha J F, Falcao A, Santos A, Pinto R, Lopes N, Nunes T, et al. Effect of opicapone and entacapone upon levodopa pharmacokinetics during three daily levodopa administrations. Eur J Clin Pharmacol. 2014; 70(9):1059-71.
60. Almeida L, Rocha J F, Falcao A, Palma P N, Loureiro A I, Pinto R, et al. Pharmacokinetics, pharmacodynamics and tolerability of opicapone, a novel catechol-O-methyltransferase inhibitor, in healthy subjects: prediction of slow enzyme-inhibitor complex dissociation of a short-living and very long-acting inhibitor. Clin Pharmacokinet. 2013; 52(2):139-51.
61. Stocchi F, Vacca L, Ruggieri S, Olanow C W. Intermittent vs continuous levodopa administration in patients with advanced Parkinson disease: a clinical and pharmacokinetic study. Arch Neurol. 2005; 62(6):905-10.
62. Brusa L, Pierantozzi M, Bassi A, Fedele E, Lunardi G, Giacomini P, et al. Temporal administration of entacapone with slow release L-dopa: pharmacokinetic profile and clinical outcome. Neurol Sci. 2004; 25(2):53-6.
63. Myllyla V V, Sotaniemi K A, Illi A, Suominen K, Keranen T. Effect of entacapone, a COMT inhibitor, on the pharmacokinetics of levodopa and on cardiovascular responses in patients with Parkinson's disease. European Journal Clinical Pharmacology. 1993; 45:419-23.
64. Falcao A, Santos A, Ferreira J J, Lee A J, Hernandez B, Rocha J F, et al. Decision-making process for opicapone's bedtime regimen [abstract]. https://www.mdsabstracts.org/abstract/decision-making-process-for-opicapones-bedtime-regimen/. Accessed May 14, 2021. Movement Disorders. 2017;32 (Suppl 2).
65. NICE. Parkinson's disease with end-of-dose motor fluctuations: opicapone. Evidence summary [ES9] Published date: 21 Mar. 2017. Available at https://www.nice.org.uk/advice/es9/chapter/Estimated-impact-for-the-NHS.
66. de la Fuente-Fernandez R, Lu J Q, Sossi V, Jivan S, Schulzer M, Holden J E, et al. Biochemical variations in the synaptic level of dopamine precede motor fluctuations in Parkinson's disease: PET evidence of increased dopamine turnover. Ann Neurol. 2001; 49(3):298-303.
67. Stocchi F, Olanow C W. Continuous dopaminergic stimulation in early and advanced Parkinson's disease. Neurology. 2004;62(1 Suppl 1):556-63.
68. Lees A J, Ferreira J, Rascol O, Reichmann H, Stocchi F, Tolosa E, et al. Opicapone for the management of end-of-dose motor fluctuations in patients with Parkinson's disease treated with L-DOPA. Expert Rev Neurother. 2017; 17(7):649-59.
69. Fabbri M, Ferreira J J, Lees A, Stocchi F, Poewe W, Tolosa E, et al. Opicapone for the treatment of Parkinson's disease: A review of a new licensed medicine. Mov Disord. 2018; 33(10):1528-39.
70. Ferreira J J, Lees A, Rocha J F, Poewe W, Rascol O, Soares-da-Silva P, et al. Opicapone as an adjunct to levodopa in patients with Parkinson's disease and end-of-dose motor fluctuations: a randomised, double-blind, controlled trial. The Lancet Neurology. 2016; 15(2):154-65.
71. Lees A J, Ferreira J, Rascol O, Poewe W, Rocha J F, McCrory M, et al. Opicapone as Adjunct to Levodopa Therapy in Patients With Parkinson Disease and Motor Fluctuations: A Randomized Clinical Trial. JAMA neurology. 2017; 74(2):197-206.
72. Takeda A, Takahashi R, Tsuboi Y, Nomoto M, Maeda T, Nishimura A, et al. Randomized, Controlled Study of Opicapone in Japanese Parkinson's Patients with Motor Fluctuations. Mov Disord. 2021; 36(2):415-23.
73. Ferreira J J, Lees A, Rocha J F, Poewe W, Rascol O, Soares-da-Silva P. Long-term efficacy of opicapone in fluctuating Parkinson's disease patients: a pooled analysis of data from two phase 3 clinical trials and their open-label extensions. Eur J Neurol. 2019; 26(7):953-60.
74. Deane K H, Spieker S, Clarke C E. Catechol-O-methyltransferase inhibitors versus active comparators for levodopa-induced complications in Parkinson's disease. Cochrane Database Syst Rev. 2004(4):CD004553.
75. Schade S, Mollenhauer B, Trenkwalder C. Levodopa Equivalent Dose Conversion Factors: An Updated Proposal Including Opicapone and Safinamide. Movement Disorders Clinical Practice. 2020; 7(3):343-5.
76. Ferreira J J, Lees A J, Poewe W, Rascol O, Rocha J F, Keller B, et al. Effectiveness of opicapone and switching from entacapone in fluctuating Parkinson disease. Neurology. 2018;90(21):e1849-e57.
77. Vokurka P, Barron A, Sumaria S, Stockford L, Jarman P, Bhatia K, et al. Opicapone Efficacy and Tolerability in Parkinson's Disease Patients Reporting Insufficient Benefit/Failure of Entacapone. Mov Disord Clin Pract. 2020; 7(8):955-60.
78. Lees A J. Evidence-based efficacy comparison of tolcapone and entacapone as adjunctive therapy in Parkinson's disease. CNS neuroscience & therapeutics. 2008; 14(1):83-93.
79. Muller T. Catechol-o-methyltransferase inhibitors in Parkinson's disease. Drugs. 2015; 75(2):157-74.
80. Reichmann H, Lees A, Rocha J F, Magalhaes D, Soares-da-Silva P, investigators O. Effectiveness and safety of opicapone in Parkinson's disease patients with motor fluctuations: the OPTIPARK open-label study. Transl Neurodegener. 2020;9(1):9.
81. Hauser R A, Auinger P. Determination of minimal clinically important change in early and advanced Parkinson's disease. Mov Disord. 2011; 26(5):813-8.
82. Lees A, Ferreira J J, Rocha J F, Rascol O, Poewe W, Soares-da-Silva P R I. Safety Profile of Opicapone in the Management of Parkinson's Disease. J Parkinsons Dis. 2019; 9:733-40.
83. Stocchi F, Antonini A, Barone P, Tinazzi M, Zappia M, Onofrj M, et al. Early DEtection of wEaring off in Parkinson disease: the DEEP study. Parkinsonism Relat Disord. 2014; 20(2):204-11.
84. Stacy M, Bowron A, Guttman M, Hauser R, Hughes K, Larsen J P, et al. Identification of motor and nonmotor wearing-off in Parkinson's disease: comparison of a patient questionnaire versus a clinician assessment. Mov Disord. 2005; 20(6):726-33.
85. Stacy M. The wearing-off phenomenon and the use of questionnaires to facilitate its recognition in Parkinson's disease. J Neural Transm (Vienna). 2010; 117(7):837-46.
86. Colombo D, Abbruzzese G, Antonini A, Barone P, Bellia G, Franconi F, et al. The “gender factor” in wearing-off among patients with Parkinson's disease: a post hoc analysis of DEEP study. ScientificWorldJournal. 2015; 2015:787451.
87. Lees A, Ferreira J J, Rocha J F, Rascol O, Poewe W, Gama H, et al. Safety Profile of Opicapone in the Management of Parkinson's Disease. J Parkinsons Dis. 2019; 9(4):733-40.
88. G. Ebersbach, J. Ferreira, A. Antonini, A. T Santos, D. Magalhaes, J. F Rocha, P. Soares-da-Silva. Opicapone's added benefit as a first-line adjunctive therapy to levodopa and when used promptly in the motor fluctuations spectrum of Parkinson's disease: a post-hoc analysis of BIPARK-I and II [abstract]. Mov Disord. 2020; 35 (suppl 1). https://www.mdsabstracts.org/abstract/opicapones-added-benefit-as-α-first-line-adjunctive-therapy-to-levodopa-and-when-used-promptly-in-the-motor-fluctuations-spectrum-of-parkinsons-disease-a-post-hoc-analysis-of-bipark/. Accessed Apr. 19, 2021.
89. Hauser R A, Gordon M F, Mizuno Y, Poewe W, Barone P, Schapira A H, et al. Minimal clinically important difference in Parkinson's disease as assessed in pivotal trials of pramipexole extended release. Parkinsons Dis. 2014; 2014:467131.
90. Nissinen H, Kuoppamaki M, Leinonen M, Schapira A H. Early versus delayed initiation of entacapone in levodopa-treated patients with Parkinson's disease: a long-term, retrospective analysis. Eur J Neurol. 2009; 16(12):1305-11.
91. Hauser R A, Deckers F, Lehert P. Parkinson's disease home diary: further validation and implications for clinical trials. Mov Disord. 2004; 19(12):1409-13.
92. Goetz C G, Tilley B C, Shaftman S R, Stebbins G T, Fahn S, Martinez-Martin P, et al. Movement Disorder Society-sponsored revision of the Unified Parkinson's Disease Rating Scale (MDS-UPDRS): scale presentation and clinimetric testing results. Mov Disord. 2008; 23(15):2129-70.
93. Chaudhuri K R, Schrag A, Weintraub D, Rizos A, Rodriguez-Blazquez C, Mamikonyan E, et al. The movement disorder society nonmotor rating scale: Initial validation study. Mov Disord. 2020; 35(1):116-33.
94. Jenkinson C, Fitzpatrick R, Peto V, Greenhalf R, Hyman N. The PDQ-8: Development and validation of a short-form parkinson's disease questionnaire. Psychol Health. 1997; 12:805-14.
95. Guy W. Clinical global impressions. ECDEU Assessment Manual for Psychopharmacology. Rockville, MD: Department of Health, Education, and Welfare, Washington, DC.; 1976. p. 218-22.
96. Jenner P, McCreary A C, Scheller D K. Continuous drug delivery in early- and late-stage Parkinson's disease as a strategy for avoiding dyskinesia induction and expression. J Neural Transm (Vienna). 2011; 118(12):1691-702.
97. Olanow C W, Obeso J A, Stocchi F. Continuous dopamine-receptor treatment of Parkinson's disease: scientific rationale and clinical implications. Lancet Neurol. 2006; 5(8):677-87.
98. Olanow C W, Obeso J A, Stocchi F. Drug insight: Continuous dopaminergic stimulation in the treatment of Parkinson's disease. Nat Clin Pract Neurol. 2006; 2(7):382-92.
99. Picconi B, Piccoli G, Calabresi P. Synaptic dysfunction in Parkinson's disease. Adv Exp Med Biol. 2012; 970:553-72.
100. Stocchi F, Rascol O, Kieburtz K, Poewe W, Jankovic J, Tolosa E, et al. Initiating levodopa/carbidopa therapy with and without entacapone in early Parkinson disease: the STRIDE-PD study. Ann Neurol. 2010; 68(1):18-27.
101. Smith L A, Jackson M J, Al-Barghouthy G, Rose S, Kuoppamaki M, Olanow W, et al. Multiple small doses of levodopa plus entacapone produce continuous dopaminergic stimulation and reduce dyskinesia induction in MPTP-treated drug-naive primates. Mov Disord. 2005; 20(3):306-14.
102. Muhlack S, Herrmann L, Salmen S, MUller T. Fewer fluctuations, higher maximum concentration and better motor response of levodopa with catechol-O-methyltransferase inhibition. J Neural Transm (Vienna). 2014; 121(11):1357-66.
103. Kuoppamaki M, Korpela K, Marttila R, Kaasinen V, Hartikainen P, Lyytinen J, et al. Comparison of pharmacokinetic profile of levodopa throughout the day between levodopa/carbidopa/entacapone and levodopa/carbidopa when administered four or five times daily. Eur J Clin Pharmacol. 2009; 65(5):443-55.
104. Loewen G, Lewitt P, Olanow C W, Kieburtz K, Liang G, Jimenez R, et al. Pharmacokinetics of Opicapone and Effect on COMT and Levodopa Pharmacokinetics in Patients with Parkinson's Disease [abstract]. Mov Disord. 2019; 34 (suppl 2). https://www.mdsabstracts.org/abstract/pharmacokinetics-of-opicapone-and-effect-on-comt-and-levodopa-pharmacokinetics-in-patients-with-parkinsons-disease/. Accessed Mar. 15, 2021.
105. Waters C H, Kurth M, Bailey P, Shulman L M, LeWitt P, Dorflinger E, et al. Tolcapone in stable Parkinson's disease: efficacy and safety of long-term treatment. The Tolcapone Stable Study Group. Neurology. 1997; 49(3):665-71.
106. Hauser R A, Panisset M, Abbruzzese G, Mancione L, Dronamraju N, Kakarieka A. Double-blind trial of levodopa/carbidopa/entacapone versus levodopa/carbidopa in early Parkinson's disease. Mov Disord. 2009; 24(4):541-50.
107. Brooks D J, Sagar H. Entacapone is beneficial in both fluctuating and non-fluctuating patients with Parkinson's disease: a randomised, placebo controlled, double blind, six month study. J Neurol Neurosurg Psychiatry. 2003; 74(8):1071-9.
108. Poewe W H, Deuschl G, Gordin A, Kultalahti E R, Leinonen M. Efficacy and safety of entacapone in Parkinson's disease patients with suboptimal levodopa response: a 6-month randomized placebo-controlled double-blind study in Germany and Austria (Celomen study). Acta Neurol Scand. 2002; 105(4):245-55.
109. Early ParkinSon wIth L-DOPA and OpicapoNe [EPSILON] study; EudraCT number 2020-005011-52. Available at https://www.clinicaltrialsregister.eu/ctr-search/trial/2020-005011-52/CZ. Last accessed May 2021.
110. OpicApone Sleep dISorder (OASIS) study. EudraCT number 2020-001176-15. Available at https://www.clinicaltrialsregister.eu/ctr-search/trial/2020-001176-15/DE. Last accessed May 2021.
111. Storch A, Schneider C B, Wolz M, Sturwald Y, Nebe A, Odin P, et al. Nonmotor fluctuations in Parkinson disease: severity and correlation with motor complications. Neurology. 2013; 80(9):800-9.
112. Antonini A, Tinazzi M, Abbruzzese G, Berardelli A, Chaudhuri K R, Defazio G, et al. Pain in Parkinson's disease: facts and uncertainties. Eur J Neurol. 2018;25(7):917-e69.
113. Nebe A, Ebersbach G. Pain intensity on and off levodopa in patients with Parkinson's disease. Mov Disord. 2009; 24(8):1233-7.
114. OpiCapone Effect on motor fluctuations and pAiN [OCEAN] study; EudraCT number 2020-001175-32. Available at https://www.clinicaltrialsregister.eu/ctr-search/trial/2020-001175-32/PT. Last accessed May 2021.
115. Poewe W, Antonini A. Novel formulations and modes of delivery of levodopa. Mov Disord. 2015; 30(1):114-20.
116. Giladi N, Caraco Y, Gurevich T, Djaldetti R, Adar L, Rachmilewitz Minei T, et al. ND0612 (levodopa/carbidopa for subcutaneous infusion) achieves stable levodopa plasma levels when administered in low and high doses in patients with PD [abstract]. Mov Disord. 2017; 32 (suppl 2). http://www.mdsabstracts.org/abstract/nd0612-levodopacarbidopa-for-subcutaneous-infusion-achieves-stable-levodopa-plasma-levels-when-administered-in-low-and-high-doses-in-patients-with-pd/. Accessed Feb. 15, 2018.

1. A method of treating a patient with Parkinson's disease, comprising administering an effective amount of opicapone to the patient, wherein:
a) the patient is being concurrently treated with an effective amount of levodopa and an effective amount of a DDCI; and
b) the patient is without clinically diagnosed motor complications.

2. The method of claim 1, wherein the patient displays a total score of MDS-UPDRS Part IV (motor complications) A+B+C of zero.

3. The method of claim 1, wherein the patient displays two or fewer positive symptoms in the WOQ-9 that improve after the next dose of levodopa.

4. The method of claim 1, wherein the patient does not display motor complications selected from the group consisting of motor fluctuations and dyskinesia.

5. The method of claim 1, wherein the patient is administered levodopa 6 times or less per day.

6. The method of claim 1, wherein the patient is not currently being treated with a COMT inhibitor.

7. The method of claim 1, wherein the opicapone is administered once daily.

8. The method of claim 7, wherein the unit dose of opicapone is 5 to 100 mg.

9. The method of claim 7, wherein the unit dose of opicapone is 25 or 50 mg.

10. The method of claim 8, wherein the opicapone is administered more than 1 hour before or after a meal.

11. The method of claim 8, wherein the opicapone is administered more than 1 hour before or after administration of levodopa.

12. The method of claim 8, wherein the opicapone is administered at or near to bedtime.

13. The method of claim 8, wherein the treatment lasts at least 24 weeks.

14. The method of claim 8, wherein the patient with Parkinson's disease had not been assessed for or diagnosed with end-of-dose motor fluctuations

15. A method of treating motor signs and symptoms of a patient with Parkinson's disease, comprising administering an effective amount of opicapone to the patient, wherein:
a) the patient is being concurrently treated with an effective amount of levodopa and an effective amount of a DDCI; and
b) the patient is without clinically diagnosed motor complications.

16. A method of treating a patient with Parkinson's disease and insufficient control of motor signs and symptoms, comprising administering an effective amount of opicapone to the patient, wherein:
a) the patient is being concurrently treated with an effective amount of levodopa and an effective amount of a DDCI; and
b) the patient is without clinically diagnosed motor complications.
